(19) United States
(12) Patent Application Publication
(10) Pub. No.: US 2013/0342810 A1
(43) Pub. Date: Dec. 26, 2013

(54) EYEBALL BIOLOGICAL INFORMATION COLLECTION DEVICE AND METHOD FOR COLLECTING EYEBALL BIOLOGICAL INFORMATION

(71) Applicant: Seiko Epson Corporation, Tokyo (JP)

(72) Inventor: Tsutomu NISHIWAKI, Azumino (JP)

(21) Appl. No.: 13/908,353

(22) Filed: Jun. 3, 2013

(30) Foreign Application Priority Data
Jun. 4, 2012 (JP) 2012-126760
Jun. 4, 2012 (JP) 2012-126761

(51) Int. Cl. A61B 8/10; A61B 3/16

(52) U.S. Cl. CPC A61B 8/10; A61B 3/16
USPC 351/205; 600/400; 351/246

(57) ABSTRACT

According to one aspect of the invention, an eyeball biological information collection device that is arranged to be worn by a subject includes a ultrasonic sensor part and a pressing part. The ultrasonic sensor part is configured to transmit an ultrasonic wave to an eyeball of the subject and receive a reflection wave of the ultrasonic wave reflected within the eyeball at a time of use of the eyeball biological information collection device. The pressing part is configured to press the ultrasonic sensor part to eyelid of the subject at the time of use.

### CROSS-REFERENCE TO RELATED APPLICATIONS

This application claims priority to Japanese Patent Application No. 2012-126760 filed on Jun. 4, 2012 and Japanese Patent Application No. 2012-126761 filed on Jun. 4, 2012. The entire disclosure of Japanese Patent Application Nos. 2012-126760 and 2012-126761 is hereby incorporated herein by reference.

### BACKGROUND

1. Technical Field

The present invention relates to an eyeball biological information collection device and a method for collecting eyeball biological information.

2. Related Art

An eyeball of human body has a structure that vitreum and intraocular fluid are filled in a part surrounded by scleral and cornea. And, it has been elucidated that high intraocular pressure, that is, a pressure of the intraocular fluid is one of the causes of glaucoma. Therefore, when a treatment of glaucoma is performed, a test is performed by measuring changes in intraocular pressure after the treatment of medication therapy, or the like. And, the effect of the treatment was checked from the changes in intraocular pressure relative to the elapsed time.

Japanese Laid-open Patent Application No. 2008-272308 discloses a device that examines eyeball by using ultrasonic wave. According to Japanese Laid-open Patent Application No. 2008-272308, first, an operator uses an ultrasonic probe to be contacted to a test cornea. And, the ultrasonic probe transmits the ultrasonic wave and receives the reflection echo, which is reflected from eyeball. In this device, a reflected position is detected from the time that the reflection echo reaches to the probe. And, the device calculates a size of eyeball such as axial length of the eyeball based on the reflection echo.

### SUMMARY

### Problems to be Solved by the Invention

In the well-known devices to examine eyeball, a technician used the ultrasonic probe to be contacted to a test cornea, and the ultrasonic wave was transmitted to the inner portion of the test cornea. And, the ultrasonic wave reflected at each tissue of the inner portion of the test cornea was received, and the technician observed the intensity waveform of the reflection echo. While the technician was observing the reflection echo waveform, a position or an angle of the ultrasonic probe was adjusted to obtain appropriate reflection echo. Accordingly, the position or the angle of the ultrasonic probe to examine eyeball had to be under the proper condition. Otherwise, it could not be properly examined. And, when the position or the angle of the ultrasonic probe was out of the proper condition, it could not be properly examined. Therefore, it has been desired that an eyeball biological information collection device removes an unnecessary procedure of a position adjustment to a test cornea.

Also, in the well-known devices, the eyeball biological information (cornea thickness, pleural thickness, axial length, depth of the anterior chamber, lens thickness, intraocular pressure, and the like) had to be measured in a place where the devices were installed.

In this kind of devices, in the normal living conditions, it was difficult to collect information (data) of the eyeball biological information for a long-term to examine the changes. Therefore, it has been desired that an eyeball biological information collection device can easily measure the eyeball biological information for a long-term.

For example, in the treatment/diagnosis of glaucoma, it is essential to measure the intraocular pressure as the eyeball biological information of a subject. As a method for the treatment of glaucoma, the progression of visual field disorder is stopped by lowering the intraocular pressure. After the treatment or the medication, in every activity conditions of the subject during the day (wake-up, day-to-day activities, going to bed, and the like), an improvement of a therapeutic effect can be expected by grasping changes of the intraocular pressure over several days.

### Means Used to Solve the Above-Mentioned Problems

The invention is to solve at least a part of the above described problems, and it is possible to be realized as the following embodiments or applicable examples.

According to one aspect of the invention, an eyeball biological information collection device that is arranged to be worn by a subject includes a ultrasonic sensor part and a pressing part. The ultrasonic sensor part is configured to transmit an ultrasonic wave to an eyeball of the subject and receive a reflection wave of the ultrasonic wave reflected within the eyeball at a time of use of the eyeball biological information collection device. The pressing part is configured to press the ultrasonic sensor part to eyelid of the subject at the time of use.

According to another aspect of the invention, an eyeball biological information collection device that is arranged to be worn by a subject includes an ultrasonic sensor part and an elastic member. The ultrasonic sensor part is configured to transmit an ultrasonic wave to an eyeball of the subject and receive a reflection wave of the ultrasonic wave reflected at the eyeball at a time of use of the eyeball biological information collection device. The elastic member is configured on a side, which is an opposite side facing toward an eyelid of the subject, at the time of use of the ultrasonic sensor part.

According to another aspect of the invention, an eyeball biological information collection device that is arranged to be worn by a subject includes an ultrasonic sensor part and an elastic supporting member. The ultrasonic sensor part is configured to transmit an ultrasonic wave to an eyeball of the subject and receive a reflection wave reflected at the eyeball at a time of use of the eyeball biological information collection device. The elastic supporting member is configured to support the ultrasonic sensor part and extending in a direction toward an eyelid of the subject at the time of use.

According to another aspect of the invention, an eyeball biological information collection device that is arranged to be worn by a subject includes an ultrasonic sensor part, a frame, and a supporting part. The ultrasonic sensor part is configured to transmit an ultrasonic wave to an eyeball of the subject and receive a reflection wave of the ultrasonic wave reflected at the eyeball at a time of use of the eyeball biological information collection device. The frame is arranged to be worn onto an ear and nose of the subject at the time of use. The supporting part is made of an elastic material that is attached to the frame, and configured to support the ultrasonic sensor part in a direction toward an eyelid of the subject at the time of use.

According to another aspect of the invention, an eyeball biological information collection device that is arranged to be worn by a subject includes an ultrasonic sensor part, a winding part, and a pressing part. The ultrasonic sensor part is configured to transmit an ultrasonic wave to an eyeball of the subject and receive a reflection wave reflected at the eyeball at a time of use of the eyeball biological information collection device. The winding part is wound on a head region of the subject at the time of use. The pressing part is made of an elastic material that is located between the winding part and the ultrasonic sensor part, and configured to press the ultrasonic sensor part to the eyelid of the subject.

According to another aspect of the invention, an eyeball biological information collection device that is arranged to be worn by a subject includes an ultrasonic sensor part, an contacting part, a data computing part, a data memory, a timer part, and a controller. The ultrasonic sensor part is configured to transmit an ultrasonic wave to an eyeball of the subject and receive a reflection wave of the ultrasonic wave reflected at the eyeball at a time of use. The contacting part contacts tightly the ultrasonic sensor part to the eyelid of the subject at the time of use. The data computing part is configured to compute eyeball biological information based on detection data detected in the ultrasonic sensor part. The data memory part is configured to store the detection data detected in the ultrasonic sensor part and computation data computed in the data computing part. The timer part is configured to set a measurement timing and a measurement interval based on time information. The controller is configured to control the ultrasonic sensor part, the data computing part, the data memory part, and the timer part. The data computing part is configure to compute the biological information of the eyeball based on the detection data detected at the measurement timing and the measurement interval.

According to another aspect of the invention, an eyeball biological information collection method for obtaining eyeball biological information in a state in which an ultrasonic sensor part is worn on a head region of a subject includes transmitting and receiving an ultrasonic wave for an eyeball in a predetermined measurement timing and a predetermined measurement interval from the ultrasonic sensor part that is contacted on an eyelid of the subject; and computing the eyeball biological information based on a detection data detected in the ultrasonic element.

### BRIEF DESCRIPTION OF THE DRAWINGS

Referring now to the attached drawings which form a part of this original disclosure:

FIGS. 1A and 1B are related to the first embodiment; FIG. 1A is a schematic front view showing a configuration of an eyeball biological information collection device; and FIG. 1B is a schematic sectional side view to explain a relationship between an ultrasonic sensor part and an eyeball;

FIG. 2A is a schematic perspective illustration showing a configuration of an ultrasonic sensor part; FIG. 2B is schematic sectional side view showing a configuration of an ultrasonic transmitter; and FIG. 2C is a schematic sectional side view showing a configuration of an ultrasonic receiver;

FIG. 3 is an electric control block diagram of an eyeball biological information collection device;

FIGS. 4A-4D are schematic diagrams to explain a measurement procedure;

FIGS. 5A-5C are related to the second embodiment; FIG. 5A is a schematic plain view of a circuit substrate; FIG. 5B is an electric block diagram of an ultrasonic transmitter; and FIG. 5C is an electric block diagram of an ultrasonic receiver;

FIGS. 6A-6C are related to the third embodiment; FIG. 6A is a schematic front view showing a configuration of an eyeball biological information collection device; FIG. 6B is a schematic top view showing a configuration of the eyeball biological information collection device; and FIG. 6C is a schematic sectional side view to explain a relationship between the ultrasonic sensor part and the eyeball;

FIGS. 7A-7C are related to the fourth embodiment; FIG. 7A is a schematic front view showing a configuration of an eyeball biological information collection device; FIGS. 7B and 7C are a schematic sectional side view to explain a relationship between an ultrasonic sensor part and an eyeball;

FIG. 8 is related to the fifth embodiment, and is an electric block diagram of an ultrasonic sensor part;

FIGS. 9A-9C are related to the sixth embodiment, and are schematic planar views to explain an arrangement of ultrasonic wave elements;

FIG. 10 is a block diagram showing a functional constitution of an intraocular pressure measurement device of the seventh embodiment;

FIG. 11 is a schematic diagram showing an example of the intraocular pressure measurement device of the seventh embodiment;

FIG. 12 is a schematic cross-sectional view to explain positions of an ultrasonic sensor part, eyelid and eyeball of the seventh embodiment;

FIG. 13 is a schematic cross-sectional view showing a constitution of the ultrasonic sensor part of the seventh embodiment;

FIG. 14 is a flowchart of an intraocular pressure measurement in the seventh embodiment;

FIG. 15 is a flowchart showing a calibration value setting process in the intraocular pressure measurement of the seventh embodiment;

FIG. 16 is a flowchart showing a measurement process in the intraocular pressure measurement of the seventh embodiment;

FIG. 17 is a flowchart showing a calculation process of a thickness of scleral and the intraocular pressure in the intraocular pressure measurement of the seventh embodiment;

FIG. 18 is a chart showing a relationship between an intraocular pressure and a thickness of scleral by positions;

FIGS. 19A and 19B are explanatory diagrams when a calculation process for a thickness of scleral is performed;

FIG. 20 is a block diagram showing a functional constitution of an intraocular pressure measurement device of the eighth embodiment; and

FIG. 21 is a schematic cross-sectional view to explain positions of an ultrasonic sensor part, eyelid, and eyeball of the eighth embodiment.

### DETAILED DESCRIPTION OF EXEMPLARY EMBODIMENTS

In the present embodiment, it will explain about characteristic examples of an eyeball biological information collection device and a collection method for collecting eyeball biological information by using the eyeball biological information collection device. The eyeball biological information indicates corneal thickness or thickness of sclera, dimension of eyeball, intraocular pressure, lens thickness, and the like. Hereinafter, the embodiments will be explained in reference to the drawings. Also, in each drawing, each element is drawn in different scale to achieve recognizable size on each drawing.

### First Embodiment

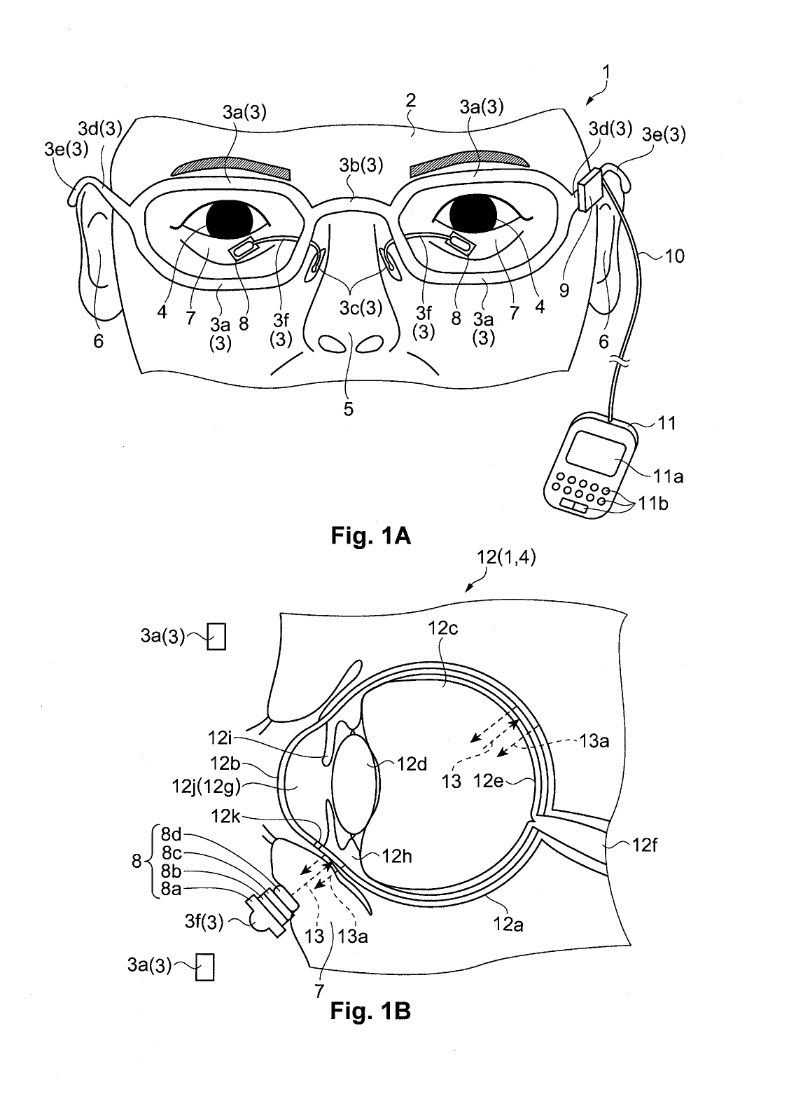

An eyeball biological information collection device related to the first embodiment will be exampled in reference to FIGS. 1A-1B to FIGS. 4A-4C. FIG. 1A is a schematic front view showing a configuration of the eyeball biological information collection device, and FIG. 1B is a schematic sectional side view to explain a relationship of an ultrasonic sensor part and the eyeball.

As shown in FIG. 1A, the eyeball biological information collection device 1 is used to be placed on a head region 2 of a subject. The eyeball biological information collection device 1 has a supporting main body part 3 as a frame. The supporting main body part 3 has the same shape as the eyeglass frame. In the supporting main body part 3, a pair of frames 3a is provided opposed to eyes 4 of the subject. The frames 3a have a shape that surrounds the eyes 4 of the subject when the head region 2 of the subject is viewed from the face side. In a case of eyeglasses, lenses are placed inner side of the frames 3a, but in the eyeball biological information collection device 1, the existence or non-existence of lenses is not particularly limited.

A bridge piece 3b is bridged between a pair of the frames 3a. And, nose pieces 3c are provided in a bridge piece 3b side of each frame 3a, and the nose pieces 3c contact sides of nose 5 of the subject. The nose pieces 3c support the eyeball biological information collection device 1.

Bows 3d are extended toward the ears 6 of the subject in opposite side of the bridge piece 3b in each frame 3a. And, wearing parts 3e are provided from upper side to back side of the ears of the subject in the bows 3d. The supporting main body part 3 is placed on the head region 2 of the subject and the wearing parts 3e are hooked on the ears 6 of the subject. Therefore, the nose pieces 3c and the wearing parts 3e contact with the head region 2 of the subject, and the supporting main body part 3 is placed on the head region 2 of the subject.

In the inner side of each frame 3a, a sensor supporting part 3f is provided as an elastic supporting part and a supporting part that extend in a direction toward bottom lid 7 of the subject as eyelid from the vicinity of the nose pieces 3c. An ultrasonic sensor part 8 is located in one end of the sensor supporting part 3f, and the sensor supporting part 3f supports the ultrasonic sensor part 8. And, the ultrasonic sensor parts 8 are provided to contact with the bottom lids 7 of the subject.

The sensor supporting part 3f has elasticity, and the sensor supporting part 3f positions the ultrasonic sensor part 8 to the bottom lid 7 of the subject. Also, when a strong power applies to the ultrasonic sensor part 8, the sensor supporting part 3f becomes bendable. Therefore, it prevents the bottom lid 7 of the subject from applying excess stress. The material of the sensor supporting part 3f can be a material that has elasticity and is settable to bend at a predetermined yield point. A metal can be used for a material of the sensor supporting part 3f, and specifically, a spring steel or a bainite steel is preferable.

The eyeball biological information collection device 1 has an arithmetic device 9 in the bows 3d, and the arithmetic device 9 is electrically connected with the ultrasonic sensor part 8 by a wire which is not shown in the drawing. And, the arithmetic device 9 carries out variety of operation by using outputs of the ultrasonic sensor part 8. In addition, the arithmetic device 9 is electrically connected with an input/output device 11 by a wire 10.

A display panel 11a and a keypad 11b are provided with the input/output device 11. The display panel 11a displays data, which is collected by the eyeball biological information collection device 1, or measurement conditions, or the like. A user can input a measurement condition by using the keypad 11b. The wire 10 and the arithmetic device 9 are connected through a connector. Accordingly, the input/output device 11 is detachable from the arithmetic device 9. The user connects the input/output device 11 to the arithmetic device 9 only when the data is inputted to the arithmetic device 9 or only when it is displayed. And, the input/output device 11 is normally separated from the arithmetic device 9 so that the eyeball biological information collection device 1 becomes lightweight. Therefore, it improves wearability of the eyeball biological information collection device 1 to the head region 2 of the subject.

The supporting main body part 3 is provided on the head region 2 of the subject, and the supporting main body part 3 supports the ultrasonic sensor parts 8. Thus, it becomes possible to maintain a position that the ultrasonic sensor parts 8 contact to the bottom lids 7 of the subject. Accordingly, even when the subject moves his/her head region 2, the ultrasonic sensor parts 8 maintain a position that is contacted to the bottom lids 7. Also, in the supporting main body part 3, a region surrounded by the frames 3a has a shape to go through the light so that it is possible that outside light can be incoming to the eyes 4 of the subject.

As shown in FIG. 1B, eyeball 12 making up eyes 4 of the subject has a spherical-shaped bag formed by sclera 12a and cornea 12b, and a gelatinous vitreum 12c is placed inside of the bag and the intraocular fluid 12g is filled. The sclera 12a is a white opaque hard film, and the portion is called as white part of the eye. The cornea 12b is an opaque hard film, and the portion is called as black part of the eye. Both of the sclera 12a and the cornea 12b together is called as an eye wall. A lens is provided in a region opposed to the cornea 12b, and a retina 12e is formed inside of the sclera 12a. A nerve in the eyes is formed by connecting a part of the retina 12e, and the nerve in the eyes are connected to the brain.

The incident light, which was incoming to the cornea 12b, passes the lens 12d. The lens 12d is a convex lens, and there is a function to provide an image for the incident light on the retina 12e. The retina 12e converts from the provided image to an electric signal, and the nerves in the eyes 12f transmits the electric signal converted from the image information to the brain. The brain recognizes an image by using the electric signal.

The intraocular fluid 12g is fulfilled inside of the eyeball 12. A pressure of the intraocular fluid 12g is called as a pressure inside the eye, and the internal stress of the eye wall is called as an intraocular pressure. When the pressure inside the eye becomes high, the intraocular pressure also becomes high because tension is applied to the eye wall. Accordingly, there is a correlation between the pressure inside the eye and the intraocular pressure. In the medical examination for the human body, as a value to analogize the pressure inside the eye, the intraocular pressure is measured. The pressure inside the eye is not directly used in the medical examination. Therefore, the intraocular pressure, which is widely used in the medical field, is not only the intraocular pressure as a measurement value of the eye wall, but also, indicating an actual pressure inside the eye.

A ciliary body 12h is located in a position surrounding the lens 12d in the eyeball 12, and the intraocular fluid 12g is secreted by the ciliary body 12h. An iris 12i is located in the cornea 12b side of the lens 12d, and a region between the iris 12i and the cornea 12b is called as an anterior chamber 12j. The iris 12i has an adjustment function of the light amount that goes through the lens 12d. In the anterior chamber 12j, a Schlemm's canal 12k is located in a region of the iris 12i's base in the lower side of the drawing. The intraocular fluid 12g passes the iris 12i and goes into the anterior chamber 12j. Next, it passes the Schlemm's canal 12k and discharges outside of the eyeball 12. Accordingly, the secreted amount of the intraocular fluid 12g that is secreted from the ciliary body 12h and the discharge amount of the intraocular fluid 12g that is discharged from the Schlemm's canal 12k affect to the pressure inside the eye.

When the intraocular pressure becomes high, the pressure of the intraocular fluid 12g affects to the retina 12e. Thus, it increases the probability of damage to the retina 12e, and the retinal ganglion cell becomes extinct that is one of the causes to become glaucoma. Therefore, after the medication to reduce the intraocular pressure, the changes of the intraocular pressure are measured so that the effect of the medication is confirmed. As a method for measuring the changes of the intraocular pressure, the eyeball biological information collection device 1 is used.

The intraocular pressure of healthy person is approximately 10 to 20 mmHg, and there are changes of 5 mmHg. And, during the day-to-day life, to recognize a condition that the intraocular pressure becomes high, it is necessary to continuously measure the intraocular pressure in a predetermined interval.

The ultrasonic sensor part 8 supported by the sensor supporting part 3f contacts to the bottom lid 7 of the subject. A base part 8a, a pressing part and an elastic part 8b as an elastic material, a sensor main body 8c, and an ultrasonic conductor 8d are superimposed in the ultrasonic sensor part 8 in the order from the sensor supporting part 3f. The base part 8a is fixed on the sensor supporting part 3f, and it has a structure to maintain a position of the ultrasonic sensor part 8. Also, the supporting part is configured by the sensor supporting part 3f and the elastic part 8b.

The elastic part 8b presses the ultrasonic sensor part 8 to the bottom lid 7 of the subject. The elastic part 8b has an elastic material. The elastic material is not limited as long as the materials have elasticity. It can have structural elasticity such as natural rubber, resin, silicone rubber, metal spring or porous sponge. The sensor main body 8c transmits an ultrasonic wave 13 to the scleral 12a. And, the sensor main body 8c receives a reflection wave 13a reflected at the scleral 12a. The ultrasonic conductor 8d conducts the ultrasonic wave 13 between the sensor main body 8c and the bottom lid 7 of the subject. The ultrasonic conductor 8d prevents a region between the sensor main body 8c and the bottom lid 7 of the subject from entering air. Thus, the ultrasonic conductor 8d suppresses the reflection before the ultrasonic wave 13 reaches to the scleral 12a. The materials of the ultrasonic conductor 8d are not limited as long as the materials conduct the ultrasonic wave 13 and suppress forming an air layer between the bottom lid 7 of the subject and the sensor main body 8c. Gelatinous elastic materials or adhesive materials such as silicon rubber, resin material, or the like can be used. In the present embodiment, for example, “Sonageru” manufactured by Takiron can be used.

The ultrasonic wave 13 transmitted from the ultrasonic sensor part 8 passes through the bottom lid 7 of the subject and reaches to the scleral 12a. A part of the ultrasonic wave 13 reflects on a surface of the scleral 12a in the bottom lid 7 side of the subject and proceeds to the ultrasonic sensor part 8 as a reflection wave 13a. A part of the ultrasonic wave 13 reflects on a surface of the scleral 12a in the vitreum 12c side and proceeds to the ultrasonic sensor part 8 as the reflection wave 13a.

The ultrasonic wave 13 that passed through the scleral 12a still passes through the ciliary body 12h and the vitreum 12c. And, a part of the ultrasonic wave 13 reaches to the scleral 12a in a position on the back side of the eyeball 12. And, a part of the ultrasonic wave 13 reflects on the surface of the scleral 12a in the vitreum 12c side and proceeds to the ultrasonic sensor part 8 as the reflection wave 13a. A part of the ultrasonic wave 13 reflects on the surface of the scleral 12a in the back side of the eyeball 12 and proceeds to the ultrasonic sensor part 8 as the reflection wave 13a.

Accordingly, the ultrasonic wave 13 goes across the eyeball 12a. The ultrasonic wave 13 reflects on the four surfaces of the scleral 12a and proceeds to the ultrasonic sensor part 8 as the reflection wave 13a. After the ultrasonic sensor part 8 transmitted the ultrasonic wave 13 at once, it receives four reflection waves 13a. Therefore, changes of the thickness of the scleral 12a in two places can be measured. In addition, changes of a distance between two points of the scleral 12a that the ultrasonic wave 13 passes through can be measured.

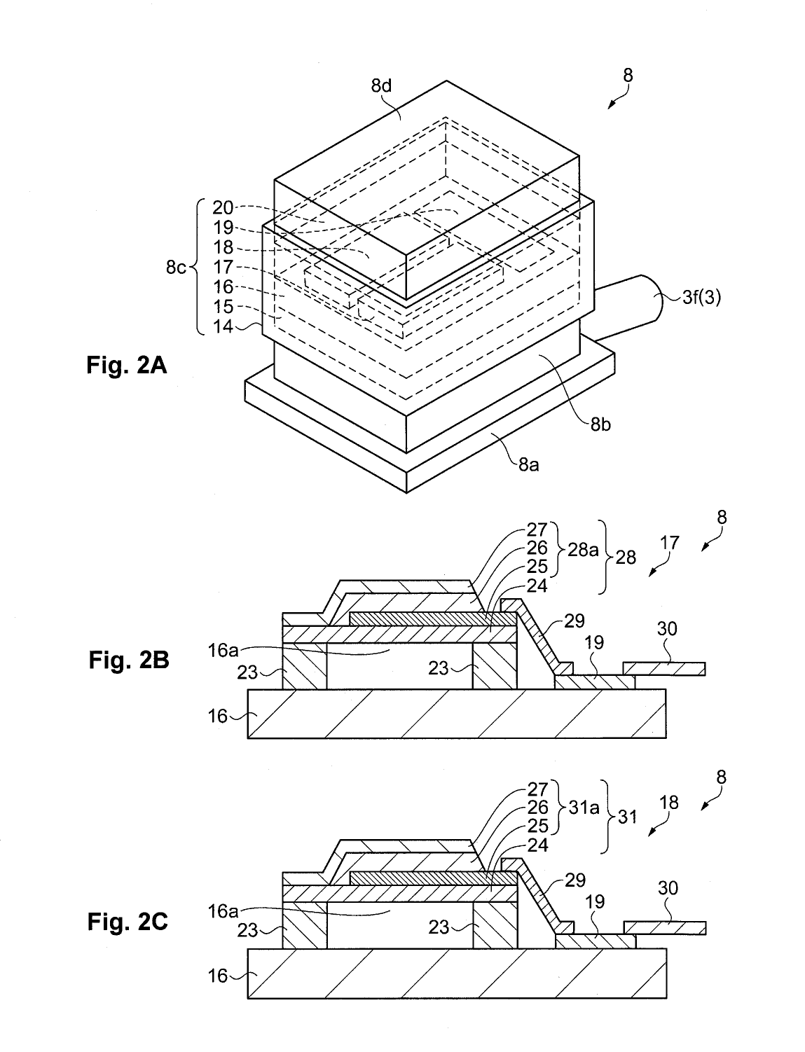

FIG. 2A is a schematic perspective illustration showing a configuration of the ultrasonic sensor part. As shown in FIG. 2A, the ultrasonic sensor part 8 has a rectangular plate-like base part 8a, and the base part 8a is fixed on the sensor supporting part 3f. The prismatic-shaped elastic part 8b is provided on a surface opposite side of the surface that connects with the sensor supporting part 3f in the base part 8a. The elastic part 8b has elasticity of extending and contracting in the up and down directions of the drawing. A material of the elastic part 8b is not particularly limited as long as it has elasticity. A silicone rubber, materials that various dopant materials are added to a natural rubber, synthetic rubber, or the like can be used. Other than that, materials that have a structural elasticity such as a coil spring, blade spring, or the like can be used. In the present embodiment, for example, the silicon rubber is used for the elastic material.

The sensor main body 8c has a box-shaped exterior part 14 that opens upper side of the drawing. The first mold 15 is provided inside the exterior part 14, and a circuit board 16 is superimposed on the first mold 15 as a substrate. The exterior part 14 is prevented from the ingress of water and dust. The first mold 15 is composed of a resin so it becomes insulation. Also, the first mold 15 has a function to fix the circuit board 16 in the exterior part 14.

An ultrasonic wave transmitter 17, an ultrasonic wave receiver 18, and a sensor circuit 19 are provided on the circuit board 16. The ultrasonic wave transmitter 17 is a part to transmit the ultrasonic wave 13. The ultrasonic wave receiver 18 is a part to receive the reflection wave 13a. And, the sensor circuit 19 is a circuit to drive the ultrasonic wave transmitter 17 and the ultrasonic wave receiver 18. The sensor circuit 19 is provided on the sensor main body 8c with the ultrasonic wave transmitter 17 and the ultrasonic wave receiver 18. Accordingly, when communicating between the sensor circuit 19 and the ultrasonic wave transmitter 17 or when communicating between the sensor circuit 19 and the ultrasonic wave receiver 18, the effect of the noise that the ultrasonic sensor part 8 receives can be suppressed.

The second mold 20 is provided to cover the ultrasonic wave transmitter 17, the ultrasonic wave receiver 18, and a sensor circuit 19 on the circuit board 16. The second mold 20 prevents the sensor main body 8c from the ingress of water and dust. The ultrasonic conductor 8d is provided to superimpose on the second mold 20 of the sensor main body 8c. The second mold 20 has a flat upper surface in the drawing so that it is possible that the ultrasonic conductor 8d is easily fixed on the sensor main body 8c.

FIG. 2B is a schematic sectional side view showing a configuration of the ultrasonic transmitter. As shown in FIG. 2B, the sensor circuit 19 is formed on the circuit board 16. The circuit board 16 is a semiconductor circuit, and the sensor circuit 19 is formed by using the publicly known photolithography method. The thickness of the circuit board 16 is not limited, but in the present embodiment, for example, it is approximately 100 μm to 150 μm. The ultrasonic wave transmitter 17 has an element substrate 23 on the circuit board 16. The circuit board 16 and the element substrate 23 are layered and it is formed on one substrate. The element substrate 23 is a semiconductor substrate. And, a part of the element substrate 23 is etched and a plurality of openings 16a is formed. The depth of the openings 16a is not limited, but in the present embodiment, for example, it is approximately 100 μm. The openings 16a are formed by the publicly known photolithography method. And, a vibrating membrane 24 is bridged on the openings of the element substrate 23. The plurality of openings 16a are arranged in an array pattern in the circuit board 16, and the vibrating membrane 24 is formed in the opening. The thickness of the vibrating membrane 24 is not particularly limited, but in the present embodiment, for example, it is approximately 0.5 μm to 4 μm. In the openings 16a of the element substrate 23, there is a space between the circuit board 16 and the vibrating membrane 24. Because of this, the vibrating membrane 24 has a beam structure in both ends so that the structure allows vibrating easily. The material of the vibrating membrane 24 is not particularly limited, but in the present embodiment, for example, a material that ZrO₂ film is provided on a plate of SiO₂ is used. It is possible to use the publicly known photolithography method and the etching method for the method for forming the element substrate 23 and the vibrating membrane 24 so that the explanation will be omitted.

A lower electrode 25, a piezoelectric body film 26, an upper electrode 27 are provided on the vibrating membrane 24. In detail, the lower electrode 25 is provided on the vibrating membrane 24, and the piezoelectric body film 26 is provided to cover at least a part of the lower electrode 25. In addition, the upper electrode 27 is provided at least a part of the piezoelectric body film 26. The lower electrode 25 and the upper electrode 27 are a conductive film, and a metal such as Al, Au, Cu, Ag, Ti, or the like can be used. The thickness of the lower electrode 25 and the upper electrode 27 is not particularly limited, but in the present embodiment, for example, the thickness of the lower electrode is approximately 200 nm, and the thickness of the upper electrode 27 is approximately 50 nm. The piezoelectric body film 26 can be any material that develops displacement by voltage, and in the present embodiment, for example, PZT is formed by using the sputtering method or the evaporation method. The thickness of the piezoelectric body film 26 is not particularly limited, but in the present embodiment, for example, the thickness is approximately 0.2 μm to 5 μm. An ultrasonic wave transmitting element 28 as the ultrasonic element is configured by the vibrating membrane 24, the lower electrode 25, the piezoelectric body film 26, and the upper electrode 27, and the piezoelectric element part 28a is configured by the lower electrode 25, the piezoelectric body film 26, and the upper electrode 27.

A wire 29 connects between the lower electrode 25 and the sensor circuit 19. In the same manner, the wire 29 connects between the upper electrode 27 and the sensor circuit 19. A wire bonding or a flexible tape can be used for the wire 29. The sensor circuit 19 applies voltage to the piezoelectric body film 16 via the lower electrode 25 and the upper electrode 27 through the wire 29. And, the sensor circuit 19 applies a drive waveform to the piezoelectric body film 26 so that the ultrasonic transmitter 17 transmits the ultrasonic wave 13 by vibrating the vibrating membrane 24. A wire 30 is provided in the sensor circuit 19, and the wire 30 transmits data between the sensor circuit 19 and the arithmetic device 9.

FIG. 2C is a schematic sectional side view showing a configuration of the ultrasonic receiver. As shown in FIG. 2C, the ultrasonic receiver 18 has the electric substrate 23 on the circuit board 16. The circuit board 16 and the element substrate 23 are layered so as to form a single substrate. The element substrate 23 is a semiconductor substrate. And, a part of the element substrate 23 is etched and a plurality of openings 16a is formed. And, the openings 16a are formed by using the publicly known photolithography method. The vibrating membrane 24 is bridged on the openings 16a of the element substrate 23. The plurality of openings 16a is arranged in an array pattern on the circuit board 16, and the vibrating membrane 24 is formed in the openings. In the openings 16a of the element substrate 23, there is a space between the circuit board 16 and the vibrating membrane 24. Because of this, the vibrating membrane 24 has a beam structure in both ends so that the structure allows the vibration easily. The material of the vibrating membrane 24 is not particularly limited, but in the present embodiment, for example, a material that ZrO₂ film is provided on a plate of SiO₂ is used. It is possible to use the publicly known photolithography method and the etching method for the method for forming the element substrate 23 and the vibrating membrane 24 so that the explanation will be omitted.

The lower electrode 25, the piezoelectric body film 26, and the upper electrode 17 are provided on the vibrating membrane 24. In detail, the lower electrode 25 is provided on the vibrating membrane 24, and the piezoelectric body film 26 is provided to cover at least a part of the lower electrode 25. In addition, the upper electrode 27 is provided at least a part of the piezoelectric body film 26. The lower electrode 25 and the upper electrode 27 are a conductive film, and a metal such as Al, Au, Cu, Ag, Ti, or the like can be used. The piezoelectric body film 26 can be any material that develops displacement by voltage, and in the present embodiment, for example, PZT is formed by using the sputtering method or the evaporation method. The ultrasonic wave receiving element 31 as the ultrasonic element is configured by the vibrating membrane 24, the lower electrode 15, the piezoelectric body film 26, and the upper electrode 27. The piezoelectric element part 31a is configured by the lower electrode 25, the piezoelectric body film 26, and the upper electrode 27. Also, in the ultrasonic receiver 18, the thickness of the circuit board 16, the vibrating membrane 24, the lower electrode 25, the piezoelectric body film 26, the upper electrode 27, and the depth of the openings 16a are the same size as the ultrasonic wave transmitter 17.

The wire 29 connects between the lower electrode 25 and the sensor circuit 19. In the same manner, the wire 29 connects between the upper electrode 27 and the sensor circuit 19. A wire bonding or a flexible tape can be used for the wire 29. When the reflection wave 13a reaches to the ultrasonic receiver 18, the vibrating membrane 24 vibrates. Because of this, the piezoelectric body film 26 generates electric power, and the voltage is generated between the lower electrode 25 and the upper electrode 27. And, the sensor circuit 19 detects the voltage between the lower electrode 25 and the upper electrode 27.

The ultrasonic transmitter 17 and the ultrasonic receiver 18 are the approximately same configuration, but the ultrasonic transmitter 17 and the ultrasonic receiver 18 are respectively an independent. That is, there are the configurations that the ultrasonic transmitter 17 transmits only the ultrasonic wave 13, and the ultrasonic receiver 18 receives only the reflection wave 13a. If the ultrasonic transmitter 17 and the ultrasonic receiver 18 transmit the ultrasonic wave 13 and receive the reflection wave 13a by using a common element, a circuit that switches the signals is required. To compare to this configuration, the ultrasonic sensor part 8 has the configuration that can be easily manufactured.

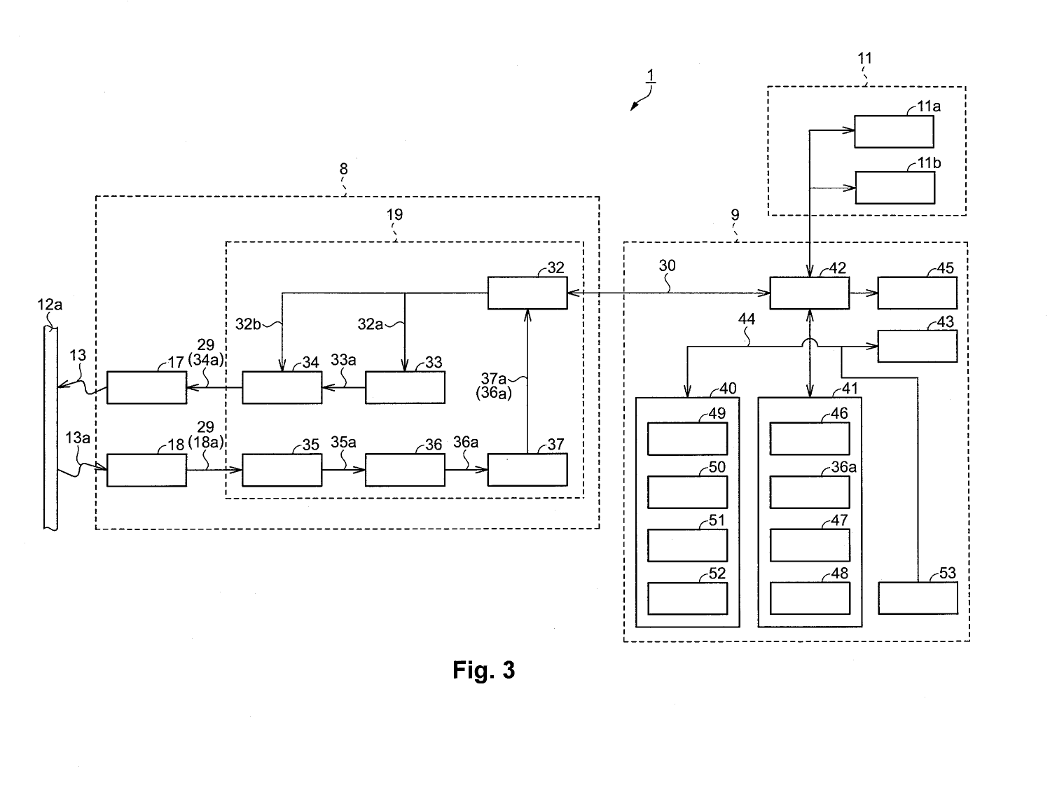

FIG. 3 is an electric control block diagram of an eyeball biological information collection device. As shown in FIG. 3, the eyeball biological information collection device 1 is mainly configured by the ultrasonic sensor part 8, the arithmetic device 9, and input/output device 11. The sensor circuit 19 in the ultrasonic sensor part 8 has a sensor controller 32. The sensor controller 32 connects to the arithmetic device 9 and performs communication with the arithmetic device 9. And, the sensor controller 32 controls operations of the ultrasonic sensor part 8.

The sensor controller 32 connects with a waveform forming part 33 and the first amplifier 34 as an amplifier circuit. The waveform forming part 33 forms the driving form 33a to drive the ultrasonic transmitter 17, and the first amplifier 34 amplifies the electric power to drive the ultrasonic transmitter 17. The sensor controller 32 controls the waveform forming part 33 to output an output command signal 32a to form a waveform. The waveform forming part 33 forms the driving waveform 33a by receiving the output command signal 32a. The waveform forming part 33 connects to the first amplifier 34, and the waveform forming part 33 outputs the driving waveform 33a to the first amplifier 34. The sensor controller 32 controls the first amplifier 34 to output a gain signal 32b to instruct gain. The first amplifier 34 inputs the driving waveform 33a and outputs the driving signal 34a that the driving waveform 33a was amplified in the gain indicated by the gain signal 32b. The first amplifier 34 is connected to the ultrasonic transmitter 17 through the wire 29, and the first amplifier 34 outputs the driving signal 34a to the ultrasonic transmitter 17.

The ultrasonic transmitter 17 applies the driving signal 34a to the ultrasonic wave transmitting element 28, and the ultrasonic wave 13 is transmitted to the scleral 12a by vibrating the vibrating membrane 24. The ultrasonic wave 13 is reflected at the scleral 12a, and the reflection wave 13a reaches to the ultrasonic receiver 13. Because of this, the vibrating membrane 24 is vibrated in the ultrasonic receiver 18, and the piezoelectric body film 26 elongates and contracts along with the vibration of the vibrating membrane 24. Because of this, the vibration of the vibrating membrane 24 is converted to the electric signal, and the converted receiving signal 18a is outputted to the second amplifier 35 as an amplifier circuit from the ultrasonic receiver 18 through the wire 29. Also, the first amplifier 34 and the second amplifier 35 are provided in the ultrasonic sensor part 8.

The second amplifier 35 amplifies the receiving signal 18a in a predetermined amplification factor. The second amplifier 35 connects to the Analog Digital converter (AD converter) 36, and the second amplifier outputs the receiving waveform 35a, that the receiving signal 18a was amplified, to the AD converter 36. The AD converter 36 converts the receiving waveform 35a to a digital receiving waveform 36a as a digital signal. The AD converter 36 connects to a memory 37, and the digital receiving waveform 36a is outputted to the memory 37. The memory 37 stores the digital receiving waveform 36a.

The memory 37 outputs an update signal 37a, which indicates that the digital receiving waveform 36a was stored, to the sensor controller 32. The sensor controller 32 communicates with the arithmetic device 9 and performs the judgment of an adequacy for sending the digital receiving waveform 36a to the arithmetic device 9. And, when it is adequate, the sensor controller 32 forwards the digital receiving waveform 36a from the memory 37 to the arithmetic device 9.

The arithmetic device 9 has a Central Processing Unit (CPU) 40 that performs various arithmetic processing as a processor, and a memory 41 as a memory part that stores variety of information. In addition, the arithmetic device 9 has an input/output interface 42 and a timer 43. The memory 41, the input/output interface 42, and the timer 43 are connected to the CPU 40 through a data bus 44. The timer 43 has time information so that the CPU can set a measurement timing based on the time information. Also, the time information is not limited to Japan Standard Time, and it can be an elapsed time after the subject wore the eyeball biological information collection device 1. And, the concept of the measurement timing includes a measurement interval.

The ultrasonic sensor part 8, the input/output device 11, and a warning part 45 are connected to the input/output interface 42. The warning part 45 is provided with the Light Emitting Diode (LED). And, the warning part 45 gets attention by blinking the light.

The concept of the memory 41 includes a semiconductor memory such as a RAM, a ROM, and the like. From a functional standpoint, a memory area such as a memory area that stores program software 46 in which the control procedure of the eyeball biological information collection device 1 is written, or a memory area that stores a digital receiving waveform 36a is set. Also, a memory area that stores a calibration value data 47 as a data to be used when the thickness of the scleral 12a is computed by using the digital receiving waveform 36a is set. In addition, a memory area that stores a measurement value data 48 as data such as an intraocular pressure value, a measurement time, and the like as a result of the computation is set. Furthermore, a memory area that functions as a work area of the CPU 40, a temporary file, and the like, and other variety of memory areas are set.

The CPU 40 performs the control of the measurement for the intraocular pressure in accordance with the program software 46 stored in the memory 41. A main controller 49 that outputs a command signal to the ultrasonic sensor part 8 to measure in a predetermined interval and acquires the digital receiving waveform 36a as a concrete function executing unit is provided. The main controller 49 displays information stored in the memory 41 in the display panel 11. And, the contents of the memory 41 are rewritten in accordance with the contents inputted from the keypad 11b.

The CPU 40 also has a relative variability value computing part 50. The relative variability value computing part 50 compares between the most updated data of the digital receiving waveform 36a and the measurement data immediately before the updated data. And, a time interval that the reflection wave 13a reaches to the ultrasonic sensor part 8 is computed based on the changes of the digital receiving waveform 36a.

Also, the CPU 40 has a film thickness computing part 51. The film thickness computing part 51 computes thickness changes of the scleral 12a by using the time interval that the computed reflection wave 13a reaches to the ultrasonic wave sensor part 8, and the calibration value data 47.

Also, the CPU 40 has an intraocular pressure value computing part 52. The intraocular pressure value computing part 52 computes changes of the intraocular pressure by using the computed thickness changes of the scleral 12a and the calibration value data 47. And, the measured time and the computed intraocular changes are stored in the memory 41 as the measurement value data 48.

Also, in the present embodiment, the above respective functions are executed in the program software by using the CPU 40. When the above respective functions can be executed by an independent electronic circuit (hardware) that does not use the CPU 40, it is possible to use such the electronic circuit.

The arithmetic device 9 also has a power source part 53. The power source part 53 has an electric storage device, and the necessary electric power in the measurement for a predetermined term is stored. When the electric power is lower than the judgment value, the power source part 53 outputs a signal to the CPU 40 to inform that the electric power is lowered. And, the main controller 49 outputs a signal to the warning part 45 to get attention.

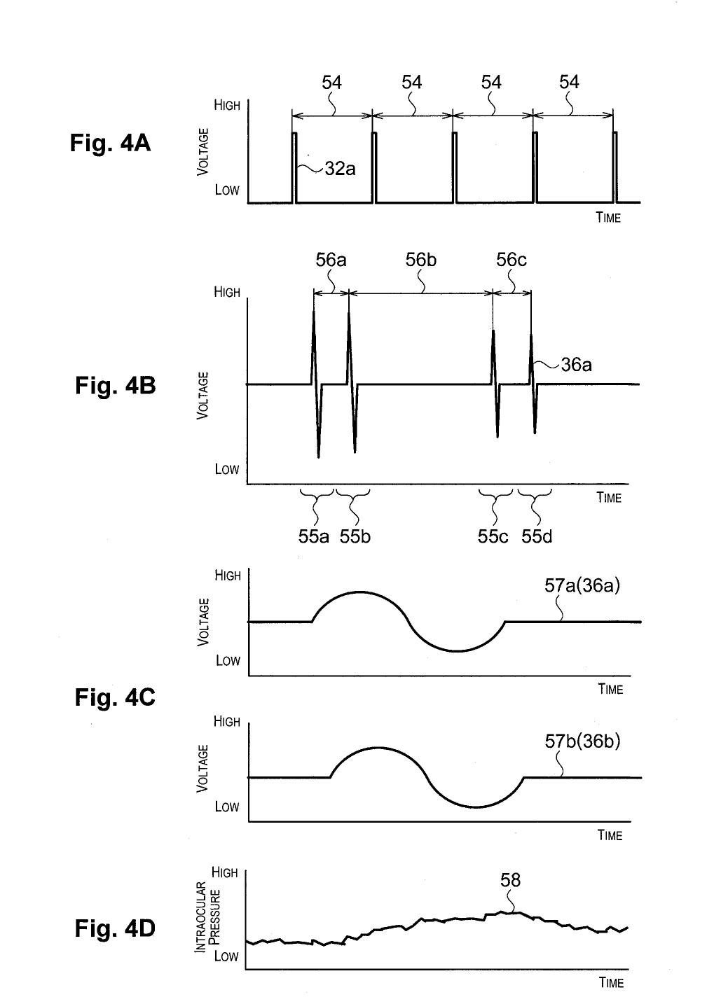

Next, a measurement procedure that the eyeball biological information election device 1 measures an intraocular pressure will be explained. FIGS. 4A-4D are schematic diagrams to explain the measurement procedure. The vertical axis of FIG. 4A to 4C indicates a voltage and the horizontal axis indicates the passage of time. First, the main controller 49 obtains the data of the measurement interval from the memory 41. The interval time is the data which is preliminary set by a user in the input/output device 11. Next, the main controller 49 obtains the time information from the timer 43. And, every time the measurement time interval is elapsed, the main controller 49 outputs a signal that instructs a measurement to the sensor controller 32.

The sensor controller 32 inputs a signal that instructs a measurement from the main controller 49 and outputs the output command signal 32a to the waveform forming part 33. As shown in FIG. 4A, the output command signal 32a is a pulse signal that rises up in every measurement interval 54.

The waveform forming part 33 forms the driving waveform 33a at a timing that inputs the output command signal 32a, and outputs it to the first amplifier 34. The first amplifier 34 amplifies the driving waveform 33a, and the amplified driving single 34a is outputted to the ultrasonic transmitter 17. The ultrasonic transmitter 17 drives the ultrasonic transmitting element 28 by the driving signal 34a, and outputs the ultrasonic wave 13 to the scleral 12a.

The ultrasonic wave 13 is reflected at the scleral 12a, and the reflection wave 13a is reflected from the scleral 12a. And, the ultrasonic receiver 18 receives the reflection wave 13a. Next, the ultrasonic receiver 18 outputs the receiving signal 18a that the reflection wave 13a is changed to an electric signal to the second amplifier 35. The second amplifier 35 amplifies the receiving signal 18a, and outputs the amplified receiving waveform 35a to the AD converter 36. The AD converter 36 converts the receiving waveform 36 to the digital signal, and the converted digital signal is stored in the memory 37.

Next, the sensor controller 32 forwards the digital receiving waveform 36a to the memory 41. Subsequently, the relative variability value computing part 50 computes a time that the ultrasonic sensor part 8 received the reflection wave 13a by using the digital receiving waveform 36a. As shown in FIG. 4B, in the digital receiving waveform 36a includes four reflection waveforms. The first reflection waveform 55a is a waveform that corresponds to the reflection wave 13a reflected at the bottom lid 7 side surface of the scleral 12a of the subject. The second reflection waveform 55b is a waveform that corresponds to the reflection wave 13a reflected at the vitreum 12c side surface in the scleral 12a of the subject that is vicinity of the bottom lid 7. The first reflection waveform 55a and the second reflection waveform 55b correspond to the reflection waves 13a that are reflected at the front and back surfaces of the scleral 12a in a place close to the bottom lid 7 of the subject.

The third reflection waveform 55c is a waveform that corresponds to the reflection wave 13a reflected at the vitreum 12c side surface of the scleral 12a at a position of the back side of the eyeball 12. The fourth reflection waveform 55c is a waveform that corresponds to the reflection wave 13a reflected at a surface in a direction toward the back of the head of the human body in the scleral 12a at a position of the backside of the eyeball 12. The third reflection waveform 55c and the fourth reflection waveform 55d correspond to the reflection waves 13a reflected at the front and back surfaces of the scleral 12a in a place of the back side of the eyeball 12.

In the digital receiving waveform 36a, an interval between the first reflection waveform 55a and the second reflection waveform 55b is the first time interval 56a. An interval between the second reflection waveform 55b and the third reflection waveform 55c is the second time interval 56b. An interval between the third reflection waveform 55c and the fourth reflection waveform 55d is the third time interval 56c. The relative variability value computing part 50 computes the first time interval 56a, the second time interval 56b and the third time interval 56c.

When the relative variability value computing part 50 computes the first time interval 56a, the second time interval 56b, and the third time interval 56c from the digital receiving waveform 36a, a phase tracking method is used. In FIG. 4C, a new reflection waveform 57a and a reference reflection waveform 57b are both one example of the digital receiving waveform 36a. The new reflection waveform 57a is the digital receiving waveform 36a as a computing target, and the reference reflection waveform 57b is the digital receiving waveform 36a that is obtained in the measurement one before the measurement that the new reflection waveform 57a was obtained. Accordingly, it means that the new reflection waveform 57a was obtained in the next measurement after the reference reflection waveform 57b was obtained.

The relative variability value computing part 50 computes the phase difference between the new reflection waveform 57a and the reference reflection waveform 57b by using the method of least squares. And, the change in time between the new reflection waveform 57a and the reference reflection waveform 57b is computed by converting the phase difference to the time. This computation is performed for the first reflection waveform 55a to the fourth reflection waveform 55d. And, by correcting the change in time computed for the first time interval 56a, the second time interval 56b, and the third time interval 56c in the reference reflection waveform 57b, the first time interval 56a, the second time interval 56b, the third time interval 56c in the new reflection waveform 57a are computed. In view of the discussion mentioned above, the concept about the phase tracking method was described, but the phase tracking method is well known computing method so that the detailed explanation was omitted.

Next, the film thickness computing part 51 inputs a scleral propagation velocity that is a propagation velocity of the ultrasonic wave 13, which proceeds in the scleral 12a from the memory 41. Also, the film thickness computing part 51 inputs a vitreum propagation velocity that is a propagation velocity of the ultrasonic wave 13, which proceeds in the vitreum 12c from the memory 41. Also, the propagation velocities are one of the calibration values stored in the memory 41. The film thickness computing part 51 computes the thickness of the scleral 12a in a place close to the bottom lid 7 of the subject by multiplying the first time interval 56a and the scleral propagation velocity. In the same manner, the film thickness computing part 51 computes the thickness of the scleral 12a in a place of the back of the eyeball 12 by multiplying the third time interval 56c and the scleral propagation velocity. In addition, the film thickness computing part 51 computes the size of the eyeball 12 by multiplying the second time interval 56b and the vitreum propagation velocity. In the size of the eyeball 12, a measurement position is not accurately set so that it is a content that measures the change relative to the passage of time.

Next, the intraocular pressure computing part 52 inputs a scleral intraocular pressure conversion data that indicates a relationship between the thickness of the scleral 12a and the intraocular pressure from the memory 41. The scleral intraocular pressure conversion data is one of the calibration values stored in the memory 41. Next, the intraocular pressure computing part 52 computes the intraocular pressure by using the value being calculated for the thickness of the scleral 12a and the scleral intraocular pressure conversion data. The vertical axis of FIG. 4D indicates the intraocular pressure, and the horizontal axis indicates the passage of time. As a result, an intraocular pressure transition line 58 is computed as shown in FIG. 4D. The output command signal 32a is outputted in every measurement interval 54 so that the measurement value of the intraocular pressure is computed in every measurement interval 54. Accordingly, the operator can observe the transition of the intraocular pressure as shown by the intraocular pressure transition line 58.

As described above, in the present embodiment, it has the following effects.

(1) According to the present embodiment, the eyeball biological information collection device 1 has the ultrasonic sensor parts 8. The ultrasonic wave 13 emitted from the ultrasonic sensor parts 8 reflects at the front surface or the back surface of the scleral 12a. The eyeball 12 is a spherical shape, and the ultrasonic wave 13 reflects at the several places in the scleral 12a. Therefore, the thickness of the scleral 12a or information of the eyeball can be measured.

(2) According to the present embodiment, the eyeball biological information collection device 1 has the supporting main body part 3, and the supporting main body part 3 supports the ultrasonic sensor parts 8. And, the elastic parts 8b are provided with the supporting main body part 3, and the elastic parts 8b press the ultrasonic sensor parts 8 to the bottom lids 7 of the subject. Because of this, the ultrasonic sensor parts 8 provided on the head region 2 of the subject through the supporting main body part 3 is pressed to the bottom lids 7 of the subject. Accordingly, even when the subject moves the head region 2, the ultrasonic sensor parts 8 contact to the bottom lids 7 of the subject so that the ultrasonic sensor parts 8 can emit the ultrasonic wave 13 to the eyeball 12 and can receive the reflection wave 13a. As a result, even when the subject moves the head region 2, the eyeball biological information collection device 1 can measure the information of the eyeball 12.

(3) According to the present embodiment, the sensor circuit part 19 is provided in the ultrasonic sensor part 8 with the ultrasonic transmitter 17 and the ultrasonic receiver 18. Accordingly, when communicating between the sensor circuit part 19 and the ultrasonic transmitter 17, or when communicating between the sensor circuit part 19 and the ultrasonic receiver 18, the effect of noise to the ultrasonic sensor part 8 can be suppressed.

(4) According to the present embodiment, the ultrasonic transmitter 17 and the ultrasonic receiver 18 are respectively an independent. And, the ultrasonic transmitter 17 has an ultrasonic transmitting element 28 to transmit the ultrasonic wave 13, and the ultrasonic receiver 18 has the ultrasonic receiving element 31 to receive the reflection wave 13a. In a case that the ultrasonic transmitter 17 and the ultrasonic receiver 18 have a common element, a device for switching between transmitting and receiving is required. Accordingly, it can be a configuration that is easy to manufacture the eyeball biological information collection device 1 compare to a device having the common element for transmitting and receiving the ultrasonic wave 13

(5) According to the present embodiment, the ultrasonic wave transmitting element 28 and the ultrasonic wave receiving element 31 are provided in the circuit board 16. And, the circuit board 16 is the semiconductor substrate so that the ultrasonic sensor part 8 can be thin and the high rigidity. As a result, the ultrasonic sensor part 8 can be minimized so as not to feel the feeling of the foreign body even when it is used to contact to the bottom lid 7 of the subject on a daily basis.

(6) According to the present embodiment, the ultrasonic conductor 8d is provided in the ultrasonic sensor part 8. And, when the eyeball biological information collection device 1 is placed on the human body, the ultrasonic conductor 8d is located between the ultrasonic sensor part 8 and the bottom lid 7 of the subject. The ultrasonic conductor 8d conducts the ultrasonic wave 13 from the ultrasonic sensor part 8 to the bottom lid 7 of the subject so that it is prevented from reducing propagation efficiency caused by entering air gap in the propagation path.

(7) According to the present embodiment, the AD converter 36 and the memory 41 are provided in the eyeball biological information collection device 1. The AD converter 36 converts the receiving waveform 35a, which is outputted from the second amplifier, to the digital signal. The memory 41 stores the digital receiving waveform 36a. Accordingly, the eyeball biological information collection device 1 stores the data of the reflection wave 13a received in the ultrasonic sensor part 8 so that the reflection wave 13a can be analyzed.

(8) According to the present embodiment, it is possible to spend the day-to-day life in a condition that the eyeball biological information collection device 1 is placed on the head region 2 of the subject. Accordingly, the data for the changes of the intraocular pressure relative to the passage of time can be acquired.

### Second Embodiment

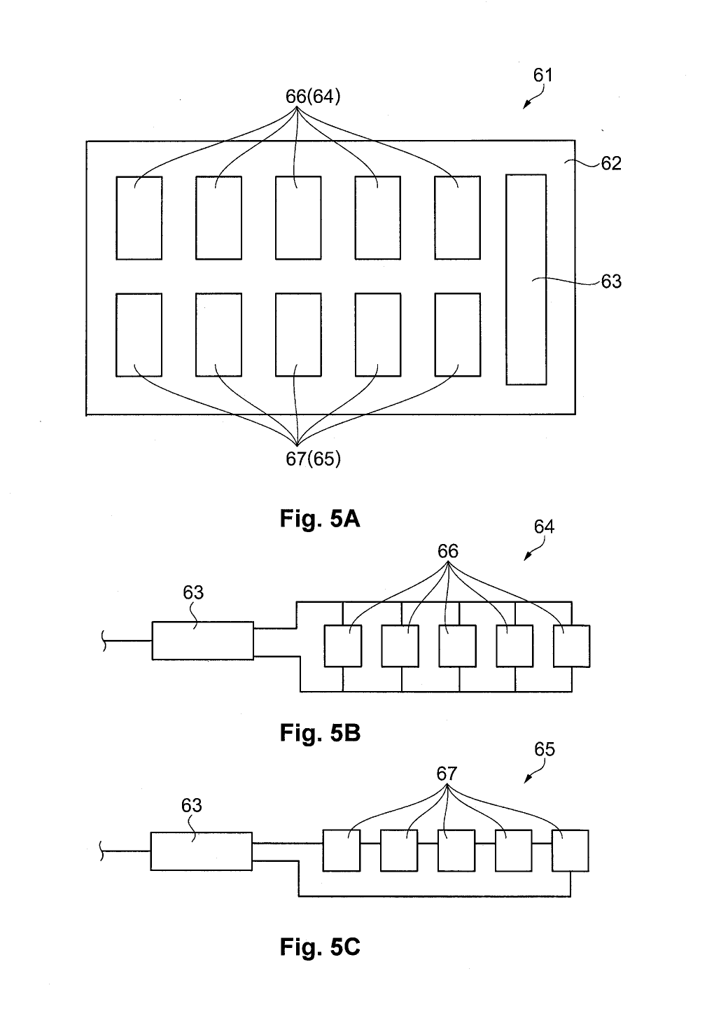

Next, one embodiment of the eyeball biological information collection device will be explained in reference to FIGS. 5A-5C. In the configuration of the present embodiment, a difference from the first embodiment is a point that the arrangements of the ultrasonic transmitter 17 and the ultrasonic receiver 18 are different. Also, the explanation about the same points in the first embodiment will be omitted.

FIGS. 5A-5C are schematic plain views of the circuit substrate. That is, in the present embodiment, as shown in FIG. 5A, an ultrasonic sensor part 61 has a circuit board 62, and a driving circuit 63 is provided on the circuit board 62. Also, an ultrasonic transmitter 64 and the ultrasonic receiver 65 are provided on the circuit board 62. And, the ultrasonic transmitter 64 has ultrasonic wave transmitting elements 66 as lined five ultrasonic elements, and the ultrasonic receiver 65 has ultrasonic wave receiving elements 67 as lined five ultrasonic elements.

FIG. 5B is an electric block diagram of an ultrasonic transmitter; and FIG. 5C is an electric block diagram of an ultrasonic receiver. As shown in FIG. 5B, the ultrasonic wave transmitting elements 66 are connected in parallel in the ultrasonic transmitter 64. Accordingly, the five ultrasonic wave transmitting elements 66 are driven by the same signal so that the ultrasonic wave 13 can be transmitted in the same waveform with the high strength. As a result, the ultrasonic sensor part 61 can receive the reflection wave 13a with the high sensitivity.

As shown in FIG. 5C, the ultrasonic wave receiving elements 67 are connected in series in the ultrasonic receiver 65. Accordingly, the ultrasonic sensor part 61 can output a signal that the outputs of the respective ultrasonic wave receiving elements 67 were added. As a result, the ultrasonic sensor part 61 can receive the reflection wave 13a with high sensitivity.

### Third Embodiment

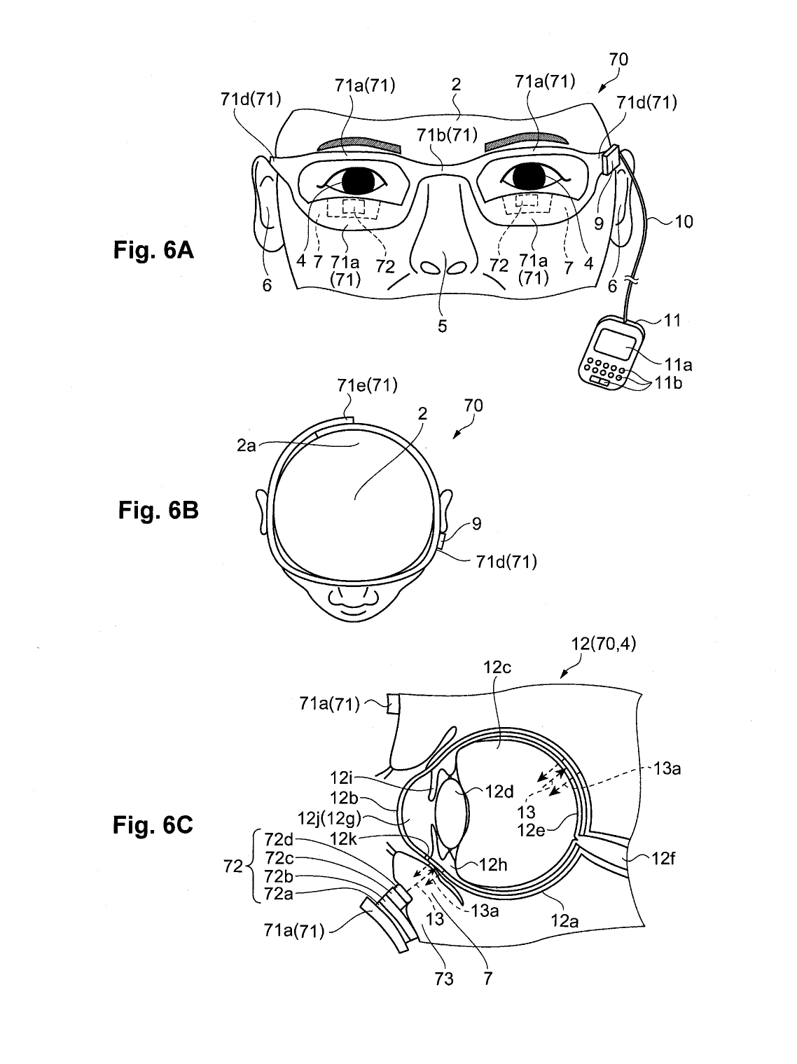

Next, one embodiment of the eyeball biology information collection device will be explained in reference to FIGS. 6A-6C. In the configuration of the present embodiment, a difference from the first embodiment is the point that the configuration of the supporting main body part 3 shown in FIGS. 1A and 1B is a mask. Also, the explanation about the same points in the first embodiment will be omitted.

FIG. 6A is a schematic front view showing a configuration of an eyeball biological information collection device. FIG. 6B is a schematic top view showing a configuration of the eyeball biological information collection device. As shown in FIGS. 6A and 6B, an eyeball biological information collection device 70 is used to set on the head part 2 of the subject. The eyeball biological information collection device 70 has a supporting main body part 71 as a winding part. The supporting main body part 71 has the same shape as a frame of an eye glass, or the supporting main body part 71 intends an eye mask part of an eye mask in which places opposed to the eyes of the subject are opened, and it is composed of fabric, rubber, elastic resin, those complexes, or the like. Also, the supporting main body part 71 has a configuration as a seat so that it is provided to contact to the head region 2 of the subject. A pair of frames 71a in a place opposed to the eyes of the subject is provided in the supporting main body part 71. The frames 71a have a shape that surrounds the eyes 4 of the subject when the head region 2 of the subject is viewed from the face side, and it is arranged to cover the bottom lids 7 of the subject.

A bridge piece 71b is bridged between a pair of the frames 71a. The bridge 71b is provided on the nose 5 of the subject so that the supporting main body part 71 is hard to move in a direction of gravitational force.

A band part 71d is extended toward the ear 6 of the subject in opposite side of the bridge piece 71b in each frame 71a. And, the band parts 71d are provided from upper side to back side of the ears of the subject. A connection part 71e is provided to connect and fix a pair of the band parts 71d at the back of the subject. The connection part 71e connects the band parts 71d with a separable function. For example, a magic tape (registered trademark) can be used for the connection part 71e. It is preferable that the band parts 71d are composed of a material having elasticity so that the supporting main body part 71 has a good wearability. For example, a threadlike rubber can be used to be knitted in the fabric.

In the frames 71a, ultrasonic sensor parts 72 are provided in a place opposed to the bottom lid 7 of the subject. The ultrasonic sensor parts 72 are provided to contact to the bottom lids 7 of the subject in a place that the frames 71a cover the bottom lids 7 of the subject. The eyeball biological information collection device 70 has the arithmetic device 9 in the band part 71d, and the arithmetic device 9 is electrically connected to the ultrasonic sensor parts 72 by a wire, which is not shown in the drawing.

FIG. 6C is a schematic sectional side view to explain a relationship between the ultrasonic sensor part and the eyeball. The ultrasonic sensor parts 72 supported by the frames 71a contact to the bottom lid 7 of the subject. The ultrasonic sensor part 72 is provided by superimposing a base part 72a, a pressing part and an elastic part 72b as an elastic member, a sensor main body 72c, and an ultrasonic conductor 72d in order from the frame 71a side. The base part 72a is fixed in the frame 71a, and it has a configuration to maintain a direction of the ultrasonic sensor part 72. A material of the elastic part 72b can be used the same material as the elastic part 8b.

The elastic part 72b presses the ultrasonic sensor part 72 to the bottom lid 7 of the subject. A part of the elastic part 72b contacts to the sensor main body 72c, and a part of it contacts to cheeks of the human body as a part of the head region 2 of the subject. Accordingly, a friction occurs between the elastic part 72b and the head region 2 of the subject so that the elastic part 72b is hard to move relative to the head region 2 of the subject. When the ultrasonic sensor part 72 moves relative to the bottom lid 7 of the subject, the noise components increase in the reflection wave 13a. On the other hand, in the present embodiment, the ultrasonic sensor part 72 is hard to move relative to the eyelid so that it can receive the reflection wave 13a that the noise generation is suppressed. The sensor main body 72c and the ultrasonic conductor 72d have the same configurations and the functions as the sensor main body 8c and the ultrasonic conductor 8d in the first embodiment so that the explanation will be omitted.

### Fourth Embodiment

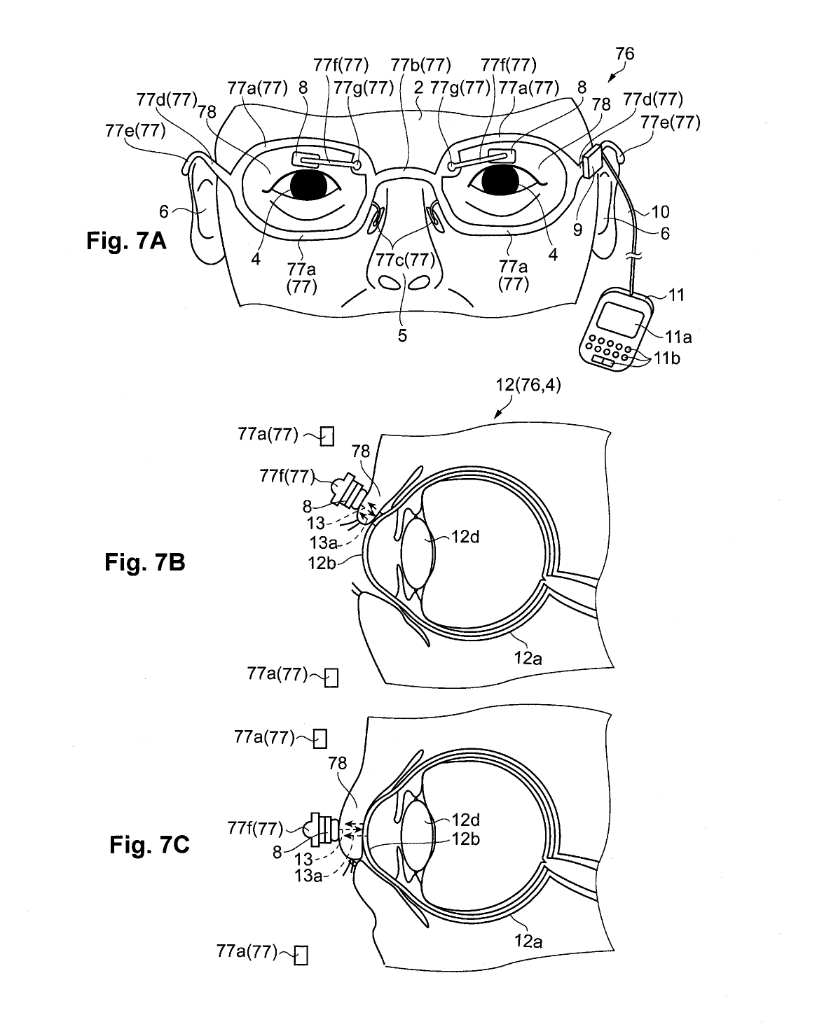

Next, one embodiment of the eye biological information collection device will be explained in reference to FIGS. 7A-7C. In the configuration of the present embodiment, a difference from the first embodiment is the point that the ultrasonic sensor part 8 contacts to the top lid of the human body. FIG. 7A is a schematic front view showing a configuration of an eyeball biological information collection device, and FIG. 7B and FIG. 7C are a schematic sectional side view to explain a relationship between an ultrasonic sensor part and an eyeball. Also, the explanation about the same points in the first embodiment will be omitted.

As shown in FIG. 7A, an eyeball biological information collection device 76 is used to be set on the head region 2 of the subject. The eyeball biological information collection device 76 has a supporting main body part 77 as a frame. The supporting main body part 77 has the same shape as a frame of an eye glass. In the supporting main body part 77, a pair of frames 77a is provided in a place opposing to the eyes 4 of the subject. The frames 77a have a shape that surrounds the eyes 4 of the subject when the head region 2 of the subject is viewed from the face side.

A bridge piece 77b is provided between the pair of the frames 77a. And, a nose piece 77c is provided in the bridge piece 77b side of each frame 77, and the nose pieces 77c contact to both sides of the nose 5 of the subject. Thus, the nose pieces 77c support the eyeball biological information collection device 76. A bow 77d is extended toward the ear 6 of the subject in opposite side of the bridge piece 77b in each frame 77a. And, wearing parts 77e are provided from upper side to back side of the ears of the subject in the bows 77d.

Sensor supporting parts 77f as an elastic supporting part and a supporting part that extend toward the top lids 78 of the human body as the eyelid from the vicinity of the nose pieces 77c inside of each frame 77a is provided. The ultrasonic sensor parts 8 are provided at one end of the sensor supporting parts 77f, and the sensor supporting parts 77f support the ultrasonic sensor parts 8. And, the ultrasonic sensor parts 8 are provided to contact to the top lids 78 of the human body.

A hinges 77g are provided in the place that the sensor supporting parts 77f connect to the frame 77a. By rotating the sensor supporting parts 77f center on the hinges 77g, it is possible that the ultrasonic sensor parts 8 move up and down in tandem with the movements of the top lids 78 of the human body.

FIG. 7B shows a condition that the top lids 78 of the human body move up and the eyes 4 of the subject is open. At this time, the ultrasonic sensor parts 8 contact to the top lids 78 of the human body, and the ultrasonic wave 13 transmitted from the ultrasonic sensor parts 8 proceeds toward the cornea 12b. And, the reflection wave 13a reflected at the cornea 12b proceeds toward the ultrasonic sensor parts 8. Accordingly, when the top lids 78 of the human body move up, the ultrasonic sensor parts 8 can measure the thickness of the cornea 12b.

FIG. 7C shows a condition that the top lids 78 of the human body move down and the eyes 4 of the subject is close. At this time, the ultrasonic sensor parts 8 contact to the top lids 78 of the human body, and it is located at a place opposing to the lens 12d. The ultrasonic wave 13 transmitted from the ultrasonic sensor parts 8 proceeds toward the cornea 12b. And, the reflection wave 13a reflected at the cornea 12b proceeds toward the ultrasonic sensor parts 8. Accordingly, when the top lids 78 of the human body move down, the ultrasonic sensor parts 8 can measure the thickness of the cornea 12b.

Accordingly, when the eyes 4 of the subject are close and also when the eyes 4 of the subject are open, the eyeball biological information collection device 76 can measure the thickness of the cornea 12b. And, in the eyeball biological information collection device 76, a corneal intraocular pressure conversion data that indicates a relationship between the thickness of the cornea 12b and the intraocular pressure is stored in the memory 41. The corneal intraocular pressure conversion data is one of the calibration value data 47 stored in the memory 41. And, the intraocular pressure value arithmetic part 52 computes the intraocular pressure by using the thickness value of the cornea 12b being calculated and the corneal intraocular pressure conversion data.

As described above, in the present embodiment, it has the following effect.

(1) According to the present embodiment, the eyeball biological information collection device 76 can measure the intraocular pressure by measuring the thickness of the cornea 12b when the eyes 4 of the subject are open and also, when the eyes 4 of the subject are close.

### Fifth Embodiment

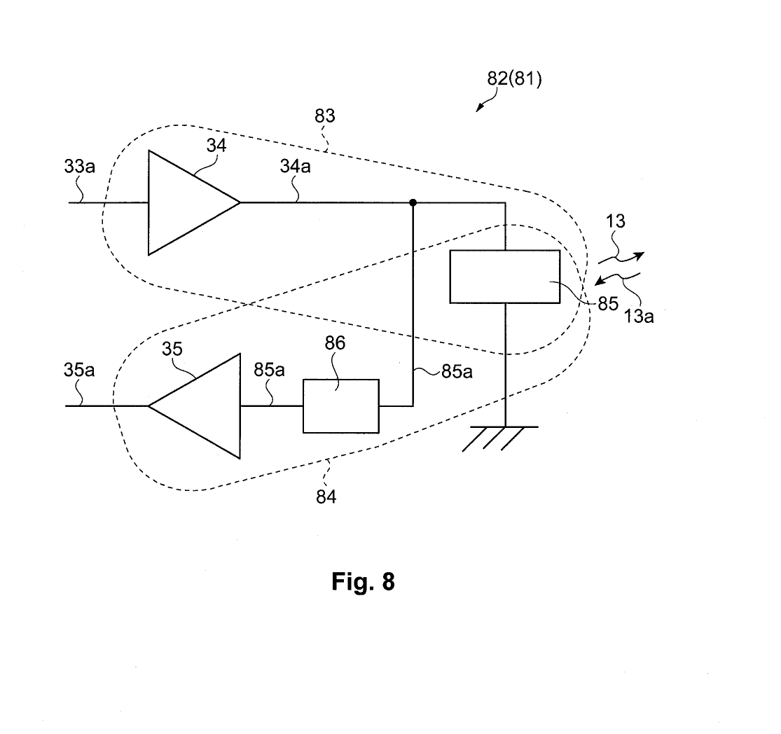

Next, one embodiment of the eyeball biological information collection device will be explained in reference to FIG. 8. In the configuration of the present embodiment, a difference from the first embodiment is the point that the ultrasonic transmitter 17 and the ultrasonic receiver 18 use a common ultrasonic wave transmitting and receiving element. FIG. 8 is an electric block diagram of the ultrasonic transmitter 17. Also, the explanation about the same points in the first embodiment will be omitted.

A shown in FIG. 8, an eyeball biological information collection device 81 has an ultrasonic sensor part 82, and the ultrasonic sensor part 82 has the ultrasonic transmitter 83 and the ultrasonic receiver 84. The ultrasonic transmitter 83 has the first amplifier 34 and an ultrasonic wave transmitting and receiving element 85 as an ultrasonic wave element. The ultrasonic transmitting and receiving element 85 has the same configuration as the ultrasonic wave transmitting element 28 and the ultrasonic receiving element 31.

The ultrasonic receiver 84 has the ultrasonic wave transmitting and receiving element 85 and the second amplifier 35, and also, a switch 86 is arranged between the ultrasonic transmitting and receiving element 85 and the second amplifier 35. When the ultrasonic sensor part 82 transmits the ultrasonic wave 13, the ultrasonic sensor part 82 switches to a condition that the switch 86 is open. Next, the ultrasonic sensor part 82 inputs the driving waveform 33a to the first amplifier 34. The first amplifier 34 amplifies the driving waveform 33a, and the amplified driving signal 34a is outputted to the ultrasonic wave transmitting and receiving element 85. The ultrasonic wave transmitting and receiving element 85 transmits the driven ultrasonic wave 13 by the driving signal 34a. Immediately after the ultrasonic wave 13 was transmitted, the ultrasonic wave transmitting and receiving element 85 switches to a condition that the switch 86 is close.

The ultrasonic wave 13 reflects at the eyeball 12, and the reflection wave 13a proceeds toward the ultrasonic sensor part 82. When the reflection wave 13a reaches to the ultrasonic wave transmitting and receiving element 85, the ultrasonic wave transmitting and receiving element 85 receives the reflection wave 13a and outputs the receiving signal 85a to the switch 86. At this point, the ultrasonic wave transmitting and receiving element 85 and the first amplifier 34 are connected, but the first amplifier 34 has high impedance so that the receiving signal 85a is not inputted to the first amplifier 34.

It is a condition that the switch 86 is close so that the receiving signal 85a is outputted to the second amplifier 35. The second amplifier 35 amplifies the receiving signal 85a, and the amplified receiving waveform 35a is outputted to the AD converter 36. Subsequent steps are the same as the first embodiment so that the explanation will be omitted.

As described above, in the present embodiment, it has the following effect.

(1) In the present embodiment, the ultrasonic wave transmitting and receiving element 85 has the function to transmit the ultrasonic wave 13 and the function to receive the reflection wave 13a. Accordingly, the ultrasonic sensor part 82 can be minimized compare to when it has an element to transmit the ultrasonic wave 13 and an element to receive the reflection wave 13a.

### Sixth Embodiment

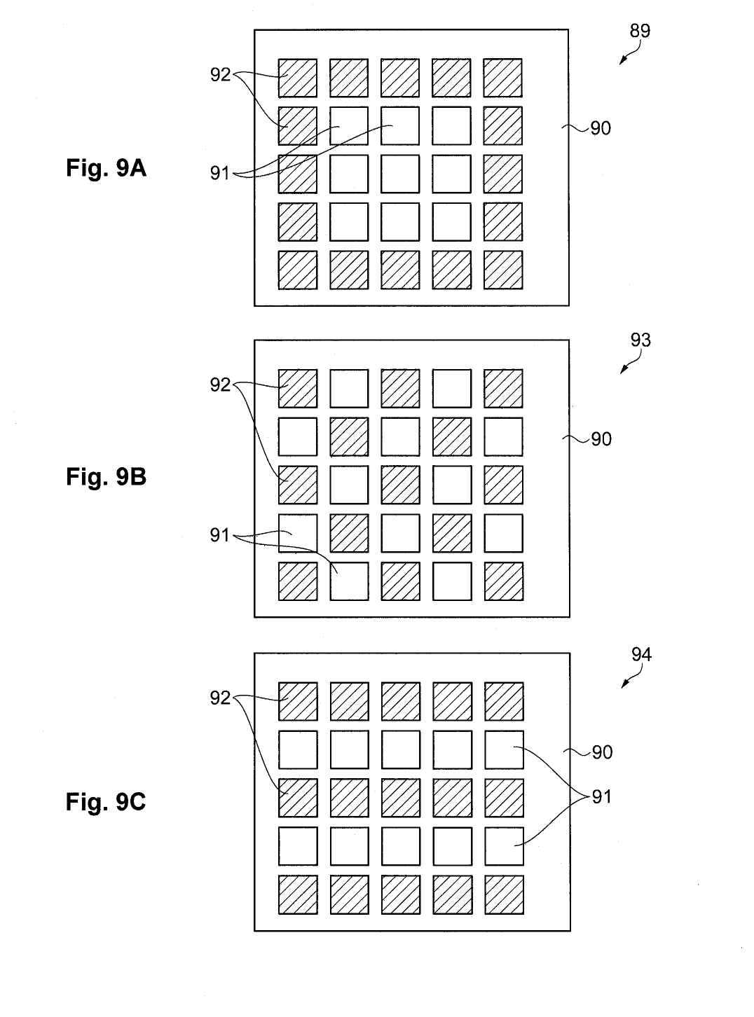

Next, one embodiment of the eyeball biological information collection device will be explained in reference to FIGS. 9A-9C. In the configuration of the present embodiment, a difference from the first embodiment is the point that the arrangement of the ultrasonic wave transmitting elements 66 and the ultrasonic wave receiving elements 67 are different. FIGS. 9A-9C are schematic planar views to explain an arrangement of ultrasonic wave elements, and the driving circuit 63 is omitted in the drawing. Also, the explanation about the same points in the first embodiment will be omitted.

As shown in FIG. 9A, the ultrasonic sensor part 89 has the circuit board 90. On the circuit board 90, the ultrasonic wave transmitting elements 91 and the ultrasonic wave receiving elements 92 as the ultrasonic wave element configure element arrays that are arranged in a matrix pattern of five rows and five columns. And, the ultrasonic wave transmitting elements 91 configure the element arrays of three rows and three columns in a central position, and the ultrasonic wave transmitting elements 91 are surrounded by the ultrasonic wave receiving elements 92. Also, the arrangement between the ultrasonic wave transmitting elements 91 and the ultrasonic wave receiving elements 92 can be reversed. It can be reversed in accordance with the transmission of the ultrasonic wave 13 and the receivability of the reflection wave 13a.

As shown in FIG. 9B, an ultrasonic sensor part 93 has the circuit board 90. On the circuit board 90, the ultrasonic wave transmitting elements 91 and the ultrasonic wave receiving elements 92 configure the element arrays that are arranged in a matrix pattern of five rows and five columns. And, the element arrays are configured such that the ultrasonic wave transmitting elements 91 and the ultrasonic wave receiving elements 92 are reciprocally arranged. Also, the arrangement between the ultrasonic wave transmitting elements 91 and the ultrasonic wave receiving elements 92 can be reversed. It can be reversed in accordance with the transmission of the ultrasonic wave 13 and the receivability of the reflection wave 13a.

As shown in FIG. 9C, an ultrasonic sensor part 94 has the circuit board 90. On the circuit board 90, the ultrasonic wave transmitting elements 91 and the ultrasonic wave receiving elements 92 configure the element arrays that are arranged in a matrix pattern of five rows and five columns. And, the ultrasonic wave transmitting elements 92 are lined in a horizon direction of the drawing, and the ultrasonic wave receiving elements 92 are lined in a horizon direction of the drawing. And, the element arrays are configured such that the lines of the ultrasonic wave transmitting elements 91 and the lines of the ultrasonic wave receiving elements 92 are reciprocally arranged in a vertical direction of the drawing. Also, the arrangement between the ultrasonic wave transmitting elements 91 and the ultrasonic wave receiving elements 92 can be reversed. It can be reversed in accordance with the transmission of the ultrasonic wave 13 and the receivability of the reflection wave 13a.

As described above, in the present embodiment, it has the following effect.

(1) According to the present embodiment, there is a configuration of the element arrays that the ultrasonic wave transmitting elements 91 and the ultrasonic wave receiving elements 92 are lined. Accordingly, transmissibility and receivability can be adjusted.

### Seventh Embodiment

In the following embodiment, for example, an intraocular pressure measurement device that measures an intraocular pressure will be explained as an eyeball biological information collection device.

### Schematic Configuration of the Intraocular Measurement Device

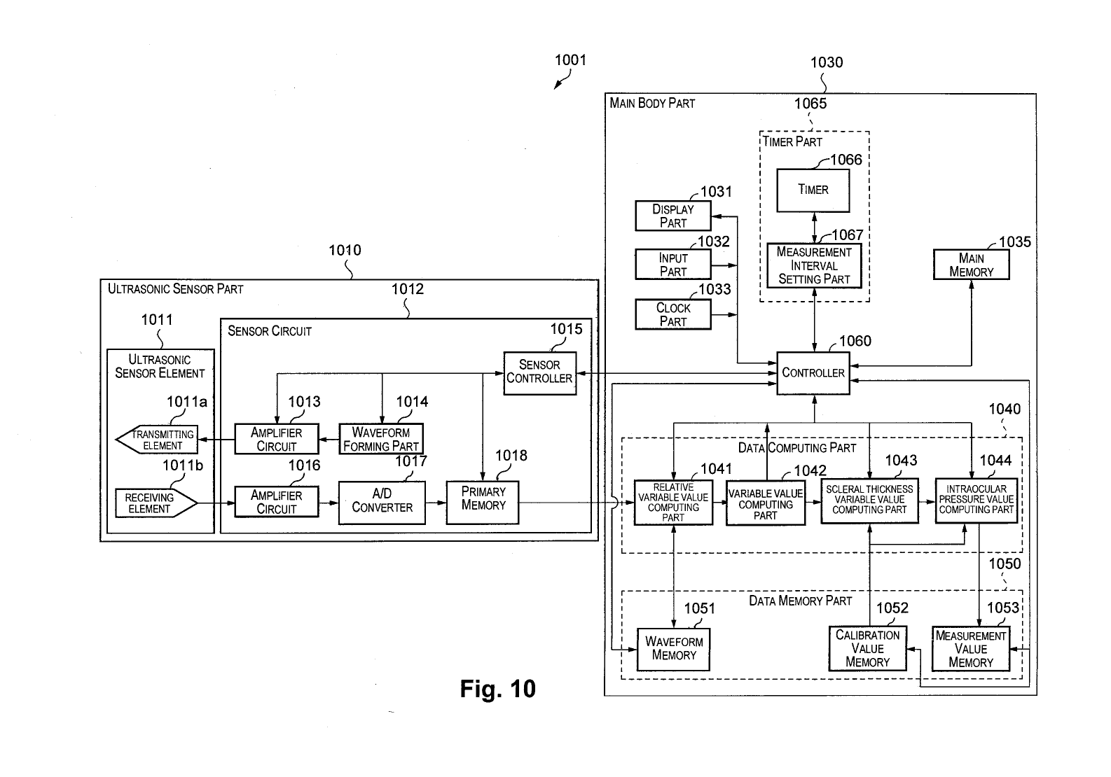

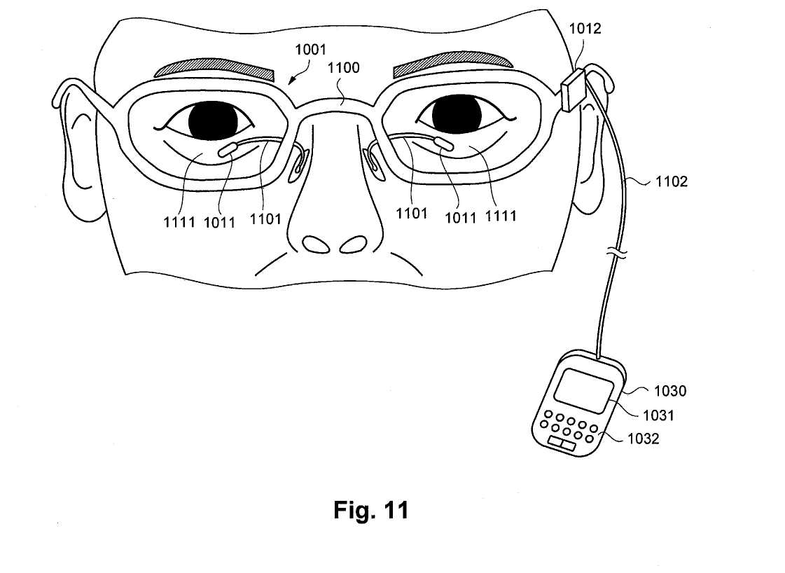

FIG. 10 is a block diagram showing a functional constitution of an intraocular pressure measurement device of the present embodiment. FIG. 11 is a schematic diagram showing an example of the intraocular pressure measurement device of the present embodiment. As shown in FIG. 10, the intraocular pressure measurement device 1001 is provided with an ultrasonic sensor part 1010 and a main body part 1030. The ultrasonic sensor part 1010 is provided with an ultrasonic element 1011 and a sensor circuit 1012. The ultrasonic element 1011 is provided with a transmitting element 1011a that transmits an ultrasonic wave and a receiving element 1011b that receives a reflection wave of an ultrasonic wave, and it is possible to be placed on the bottom lid.

The sensor circuit 1012 is provided with an amplifier circuit 1013, a waveform forming part 1014, a sensor controller 1015, an amplifier circuit 1016, an A/D converter 1017, and the primary memory 1018. The sensor controller 1015 connects to the amplifier circuit 1013 and the waveform forming part 1014, and it controls a pulse signal of the ultrasonic wave, which is transmitted from the transmitting element 1011a, and the strength. A pulse signal in a predetermined frequency is generated in the waveform forming part 1014, and the pulse signal is amplified to a predetermined strength signal in the amplifier circuit 1013, and it is inputted to the transmitting element 1011a. On the other hand, the reflection wave received in the receiving element 1011b is amplified in the amplifier circuit 1016, and it is converted from an analog signal to a digital signal in the A/D converter 1017. Once here, the received waveform data is stored in the primary memory 1018 connected to the sensor controller 1015. By the way, the ultrasonic element 1011 was explained as separate transmitting and receiving elements, but it can be a configuration that the both elements are combined. In this case, a transmitting mode and a receiving mode are switched by the time-sharing system so as to transmit and receive the ultrasonic wave.

A main body part 1030 is provided with a data computing part 1040, a data memory 1050, a controller 1060, a timer part 1065, and the like. In the data computing part 1040, a relative variability value computing part 1041, a variable value judgment part 1042, a scleral thickness variable value computing part 1043, and an intraocular pressure value computing part 1044 are connected in order, and the respective parts are connected to the controller 1060. Also, the data memory 1050 is provided with a waveform memory 1051, a calibration value memory 1052, and a measurement value memory 1053. In the waveform memory 1051, the waveform data of the received reflection waves from the front wall and the back wall of the scleral of the eyeball is stored. In the calibration value memory 1052, the respective intraocular pressures preliminary measured in at least two different postural conditions and the waveform data of the refection waves from the scleral of the eyeball measured in the intraocular pressure measurement device 1001 at the time of the postural condition, and the change ratio of the intraocular pressures relative to the thickness changes of the scleral are stored, and the data measured by using those data is used as a calibration value. In the measurement value memory 1053, the computed intraocular pressure value is stored.

In the relative variability value computing part 1041, the values of the variable waveform data of the reflection waves are computed from the waveform data of the reflection waves, which were received last time, from the front wall and the back wall of the scleral of the eyeball stored in the waveform memory 1051, and the waveform data of the reflection waves, which were received in this time, from the front wall and the back wall of the scleral of the eyeball stored in the primary memory 1018. In the variable value judgment part 1042, it judges whether the variable values computed in the relative variability value computing part 1041 are in a range of the defined value or out of the range of the defined value. By providing this kind of the variable value judgment part 1042, an error of measurements and abnormality of the measurement values can be judged, and it is possible to provide a re-measurement, a warning, or an alarm. In the scleral thickness variable value computing part 1043, the thickness of the scleral or the thickness variable value of the scleral is computed from the waveform data of the reflection waves stored in the calibration value memory 1052 and the variable values of the waveform data of the reflection waves computed in the relative variability value computing part 1041. In the intraocular pressure computing part 1044, an intraocular pressure value of the eyeball measured in this time is computed from the thickness of the scleral computed in the scleral thickness variable value computing part 1043 or the variable value of the waveform data of the reflection wave, and the intraocular pressure value stored in the calibration value memory 1052. And, the computed intraocular pressure value is stored in the measurement value memory 1053.

The timer part 1065 connects to the controller 1060, and is provided with a timer 1066 and a measurement interval setting part 1067. The measurement interval setting part 1067 sets an interval of the timer 1066, and a measurement interval to measure an intraocular pressure can be set. By providing the measurement interval setting part 1067, for example, it is possible to change the setting of the measurement interval in response to an active state of the subject. Specifically, it is possible to set the short measurement interval in the active state compare to while sleeping so that it can reduce the unnecessary measurement.

Also, the controller 1060 is connected to the aforementioned sensor controller 1015, a display part 1031, an input part 1032, a clock part 1033, and a main memory 1035. The display part 1031 is a display device that is configured by a liquid crystal panel, or the like, and it displays an intraocular pressure value or various values instructed from the controller 60. The input part 1032 is an input device that is configured by a pressing switch, or the like, and a pressing signal of the switch is outputted to the controller 1060 so that it is possible to control an input of the various data, calling data, and the like. The clock part 1033 has an oscillator and an oscillating circuit, and it is a clock device that has a clock showing time and a calendar information. The main memory 1035 is a memory device that is configured by a Read Only Memory (ROM), a Random Access Memory (RAM), and the like, and the operation program that operates the intraocular pressure measurement device 1001 is stored.

In this configuration, specifically, an intraocular pressure measurement device 1001 has a configuration as one example shown in FIG. 11. The intraocular pressure measurement device 1001 has a frame 1100 with an eye glass shape so as to wear it on the head region, and a supporting member 1101 that has elasticity and extends toward the bottom lids 1111 from the frame 1100 is provided. At a tip of the supporting member 1101, an ultrasonic element 1011 is provided, and it has a configuration that the ultrasonic element 1011 always contacts to the bottom lids 1111. A wire is provided through inside of the frame 1100 and the supporting member 1101 from the ultrasonic elements 1011 and it is connected to a sensor circuit 1012 provided in a chord section of the frame 1100. And, a cord 1102 is connected from the sensor circuit 1012, and the display 1031 and the input part 1032 are provided in the exterior part, and in the inside part, it connects to the main body part 1030 that stores the data computing part 1040.

By the way, the configuration of the above intraocular pressure measurement device 1001 is one example, so that it can be a configuration that the ultrasonic elements 1011 and the sensor circuit 1012 are arranged in a portion that contacts to the bottom lids 111, and the data computing part 1040 that computes the measurement values, the data memory 1050, the controller 1060, the main memory 1035, the timer part 1065, and the like are arranged in the chord section of the frame 1100. Also, to contact the ultrasonic elements 1011 to the eyelids, an eye mask shape, or a method for adhering it to the eyelids directly, or any methods other than the above described frame-shape can be used.

### Principle of the Intraocular Pressure Measurement

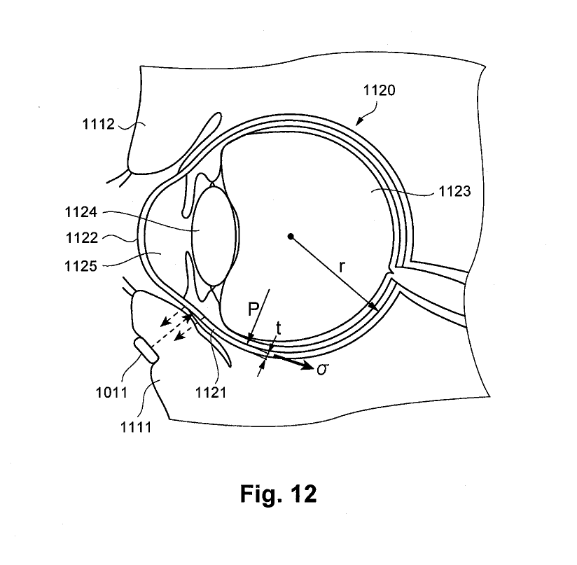

FIG. 12 is a schematic cross-sectional view to explain positions of an ultrasonic sensor part, an eyelid and an eyeball. In an eyeball 1120, the outer circumference of a vitreum 1123, a lens 1124, an anterior chamber 1125, and the like are surrounded by a film as an internal capsule. A part surrounding the anterior chamber 1125 is called as a cornea 122, and a part close to the vitreum 1124 connected from the cornea 122 is called as a scleral 1121. The scleral 1121 has a white hard film which is called as white part of the eye. In the present embodiment, the ultrasonic elements 1011 are arranged to contact to the bottom lids 1111. The ultrasonic wave is generated from the ultrasonic elements 1011, and when it reaches to the scleral 1121, the reflection waves occur at the front wall and the back wall of the scleral 1121. By detecting a receiving time lag of the reflection waves, the thickness of the scleral 1121 can be computed.

Here, if a thickness of the scleral is t, a surface stress of the scleral is σ, an intraocular pressure is P, and a radius of the eyeball is r, the following equation will be satisfied.

σ=P×r/(2t) (1)

From the equation (1), when the intraocular pressure P rises, the thickness of the scleral becomes thinner. Because of this, it is possible to assume the intraocular pressure P from the thickness t of the scleral, and it is possible to assume changes in the intraocular pressure from the changes of the thickness of the scleral.

### Configuration of Ultrasonic Sensor Part

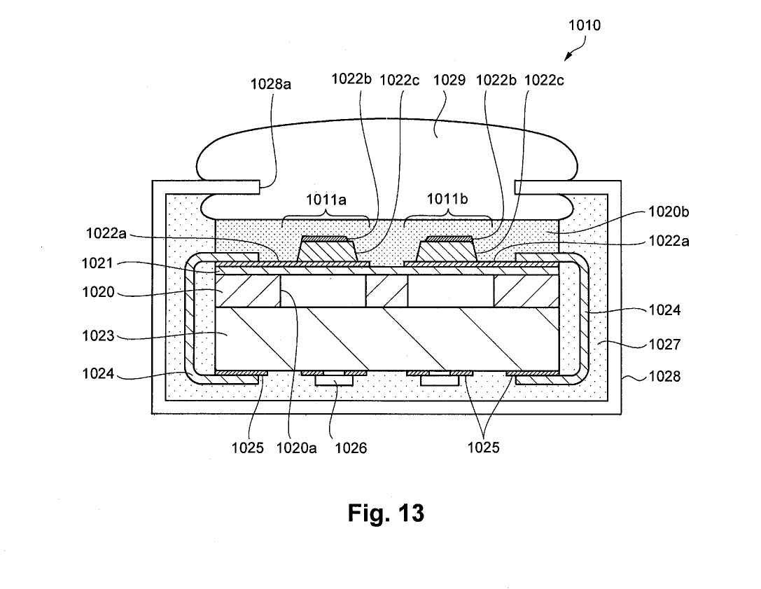

Next, one example of the configuration of the ultrasonic sensor part will be explained. In this ultrasonic sensor part, the configuration is an integrated combination of the ultrasonic element and the sensor circuit. FIG. 13 is a schematic cross-sectional view showing a constitution of the ultrasonic sensor part. The ultrasonic sensor part 1010 is provided with the transmitting element 1011a that transmits an ultrasonic wave and the receiving element 1011b that receives a reflection wave of the ultrasonic wave. These elements are arranged with plural number in an array pattern at regular intervals. The transmitting element 1011a and the receiving element 1011b have the same configuration so that the configuration of the transmitting element 1011a will be explained on behalf of these elements. The transmitting element 1011a has an opening 1020a in a substrate 1020 such as a silicon substrate, and it is provided with a vibrating membrane (membrane) 1021 to cover and to close the opening 1020a. The vibrating membrane 1021 is composed of, for example, two layers of SiO₂ layer and ZrO₂ layer. Here, in a case that the substrate 1020 is the Si substrate, the SiO₂ layer can be formed by the thermo-oxidative decomposition to the substrate surface. Also, the ZrO₂ layer can be formed on the SiO₂ layer by a method of sputtering, for example. Here, in a case that for example, PZT is used as a piezoelectric body film, the ZrO₂ layer is a layer to prevent the SiO₂ layer from dispersing Pb that constitutes the PZT. Also, the ZrO₂ layer has an effect to improve the deformation efficiency relative to the deformation of the piezoelectric body film.

A lower electrode 1022a is formed on a vibrating membrane 1021. A piezoelectric body film 1022c is formed on the lower electrode 1022a. Also, an upper electrode 1022b is formed on the piezoelectric body film 1022c. That is, there is a configuration that the piezoelectric body film 1022c is formed between the lower electrode 1022a and the upper electrode 1022b so as to configure a piezoelectric part. The piezoelectric body film 1022c is formed by forming, for example, lead zirconate titanate (PZT) in membrane. In the present embodiment, the PZT is used as the piezoelectric body film 1022c, but it can be any material if the material is shrinkable in-plane direction by applying voltage. For example, lead titanate (PbTiO₃), lead zirconate (PbZrO₃), lead lanthanum titanate ((Pb,La)TiO₃), and the like can be used. And, a protection film 1020b formed by silicone resin, or the like that covers the upper electrode 1022b of the transmitting element 1011a and the receiving element 1011b is arranged.

Also, the substrate 1020 is fixed on the base substrate 1023 formed by silicon (Si), or the like. On a surface that is opposite side of the surface fixing the substrate 1020, a sensor circuit 1012 that a circuit pattern and an integrated circuit 1026 are arranged is formed. The transmitting element 1011a and the receiving element 1011b, and the sensor circuit 1012 are connected through the flexible substrate 1024. For example, the lower electrode 1022a of the transmitting element 1011a and a connection electrode 1025 of the sensor circuit 1012 are integrally formed. Specifically, it is preferable that at least the receiving element 1011b and an amplifier circuit of the sensor circuit 1012 that connects to the receiving element are formed integrally. By this configuration, a wire with an amplifier circuit that amplifies the receiving signal in the ultrasonic element can be set short so that the effect of the nose caused by the length of the wire can be suppressed. By the way, as a configuration that forms the transmitting element 1011a and the receiving element 1011b, and the sensor circuit 1012 integrally, it can be applied by a configuration that arranges a relationship of the front and back as shown in FIG. 13, or a configuration that the transmitting element 1011a and the receiving element 1011b, and a sensor circuit 1012 are layered. Also, it can be a configuration that arranges both on one side of the substrate.

Also, the thickness of the above substrate 1020 is approximately 100 μm. The thickness of the base substrate 1023 is 100 to 150 μm. The thickness of the vibrating membrane 1021 is 0.5 to 5 μm. The thickness of the lower electrode 1022a is approximately 200 nm. The thickness of the upper electrode 1022b is 50 nm. The thickness of the piezoelectric body film 1022c is approximately 0.2 to 5 μm. Because of this, the transmitting 1011a and the receiving element 1011b can be configured thin.

The base substrate 1023 that configures the above transmitting element 1011a and the receiving element 1011b, and the sensor circuit 1012 is stored in a case 1028 and a filled resin 1027 is filled inside of the case so as to fix the base substrate. As the filled resin, an insulating resin such as epoxy resin, or the like is used, it protects the sensor circuit 1012 and prevents from the short circuit with the case 1028. By the way, it is not shown in the drawing, but a wire connecting to the sensor circuit 1012 is led to the outside of the case 1028. In addition, a viscoelastic member 1029 that contacts to the protection film 1020 protecting the transmitting element 1011a and the receiving element 1011b, and has sandwiched elasticity in an opening 1028a of the case 1028 is arranged. The viscoelastic 1029 is a polymer gel for acoustic coupling and has excellent conformable so that it has good adhesiveness to skin. And, it has acoustic impedance that is comparable with the body tissue. The viscoelastic member 1029 is the part (contacting part) tightly contacting to the eyelids in the present embodiment. By the way, in the invention, the viscoelastic member 1029 does not have to be used, and the surface of the protection film 1020b (contacting part) can tightly contact to the eyelids.

In the transmitting element 1011a of such a configuration, by applying pulsed voltage between the upper electrode 1022b and the lower electrode 1022a, the piezoelectric body film 1022c is deformed so that the vibrating membrane 1021 displaces and vibrates in a film thickness direction to generate the ultrasonic wave. The ultrasonic wave is transmitted toward the eyelids through the protection film 1020 and the viscoelastic member 1029. And, the reflection wave reflected at a border of each tissue such as the scleral 1121 of the eyeball is received in the receiving element 1011b through the viscoelastic member 1029 and the protection film 1020b. In this time, the vibrating membrane 1021 vibrates in the film thickness direction. The difference in the electrical potentials on the lower electrode 1022a side surface and the upper electrode 1022b side surface of the piezoelectric body film 1022c is generated so that a detection signal (electric current) in response to the amount of displacement of the piezoelectric body film 1022c from the upper electrode 1022b and the lower electrode 1022a is outputted.

### Measurement Procedure of Intraocular Pressure

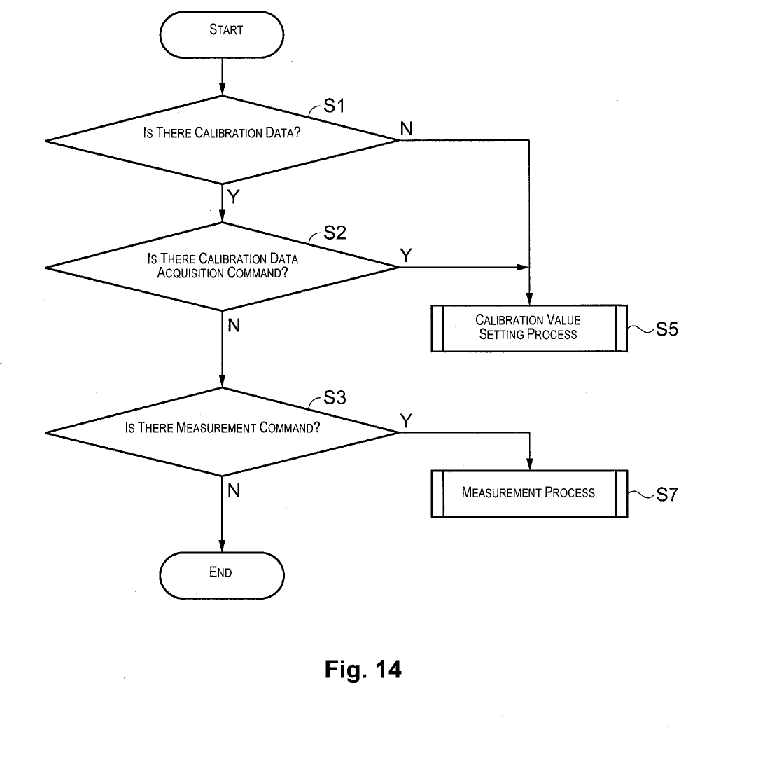

Next, the measurement procedure of the intraocular pressure in the intraocular pressure measurement device will be explained. FIG. 14 is a flowchart showing a main process flow of an intraocular pressure measurement. First, it confirms whether or not a calibration data is existed in the intraocular pressure measurement device (Step S1). Specifically, it judges whether or not the calibration data is stored in the calibration value memory 1052. If the necessary calibration data is not existed in the calibration value memory 1052, a calibration value setting process is performed in Step S5.

Next, if the calibration data is existed in the intraocular pressure measurement device, it proceeds to next step and it judges whether or not there is a calibration data acquisition command (Step S2). Here, it confirms an existence or non-existence of the calibration data required for this measurement, and for example, it determines the date that the calibration data was stored and if the calibration data was old, the calibration data acquisition command is sent. When the calibration data acquisition command was sent, it proceeds to Step S5 and the calibration value setting process is performed. When the calibration data acquisition command was not sent, it proceeds to Step S3. In Step S3, it determines whether or not there is an intraocular pressure measurement command. When the intraocular pressure measurement command was sent, it proceeds to Step S7 and performs the intraocular pressure measurement process. Also, when the intraocular pressure measurement command was not sent (at a timing that does not perform measurement), the main process is end.

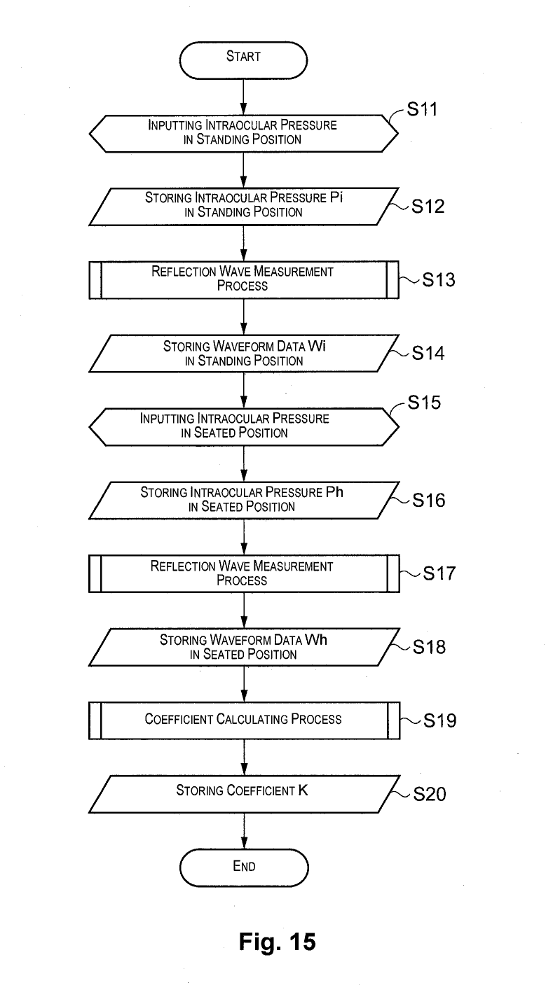

FIG. 15 is a flowchart showing one example of the calibration value setting process in the intraocular pressure measurement of the present embodiment. First, an intraocular pressure value Pi in the standing position is measured by another tonometer, and the intraocular pressure value is inputted to the intraocular pressure measurement device (Step S11). The intraocular pressure value is inputted from the input part 1032, and the intraocular pressure value Pi in the standing position is stored in the calibration value memory 1052 (Step S12). Next, in the same standing position of the above intraocular pressure measurement, a measurement for a reflection wave from the scleral of the eyeball is processed in the intraocular pressure measurement device 1001 (Step S13). And, a waveform data Wi of the reflection wave in the standing position is stored in the calibration value memory 1052 (Step S14).

Next, an intraocular pressure Ph in the seated position is measured by another tonometer, and the intraocular pressure is inputted to the intraocular pressure measurement device (Step S15). The intraocular pressure is inputted from the input part 1032, and the intraocular pressure Ph in the seated position is stored in the calibration memory 1052 (Step S16).

Next, in the same seated position of the above intraocular pressure measurement, a measurement for a reflection wave from the scleral of the eyeball is processed in the intraocular pressure measurement device 1001 (Step S17). And, a waveform data Wh of the reflection wave in the seated position is stored in the calibration value memory 1052 (Step S18).

Next, a coefficient calculating process is performed from the data stored in the above calibration value memory 1052 (Step S19). The coefficient K is stored in the calibration value memory 1052 (Step S20), and the calibration value setting process is end. Here, the coefficient K is the data indicating a change rate of the intraocular pressure relative to changes of the scleral thickness.

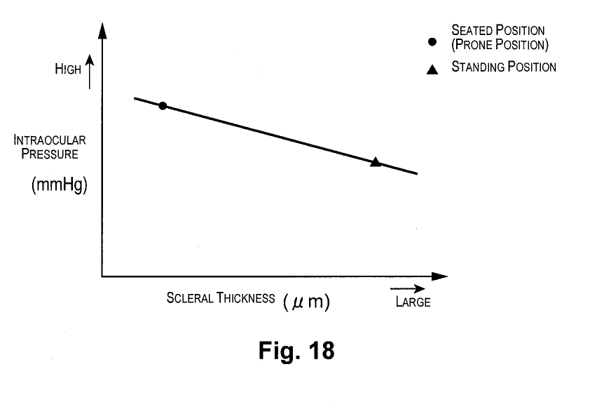

Here, the concept about the above coefficient K will be explained. It is well know that the intraocular pressure changes depending on positions and the scleral thickness of the eyeball also changes in accordance with the changes of the intraocular pressure. Because of this, if the scleral thickness of the eyeball can be determined by the changes of the intraocular pressure in different body postures, it is possible to assume an intraocular pressure from the scleral thickness by the inclination of the chart (coefficient K) indicating the intraocular pressure and the scleral thickness. For example, FIG. 18 is a chart indicating a relationship between the intraocular pressure and the scleral thickness in the positions. In this chart, it is set that the vertical axis indicates the intraocular pressure, and the horizontal axis indicates the scleral thickness. The data in the standing position and the seated position is plotted. A line connecting the values in respective positions is inclined, and by using the inclination as the coefficient K, it is possible to calculate the scleral thickness from the intraocular pressure or the intraocular presser from the scleral thickness. By the way, without calculating the thickness of the scleral, it is possible to calculate the intraocular pressure by using the waveform of the reflection wave of the scleral. Also, at least two different body postures can be employed as the positions for the calibration values. For example, the two positions can be selected from a standing position, a seated position, a prone position, a decubitus position, a dorsal position, a recumbent position, and the like.

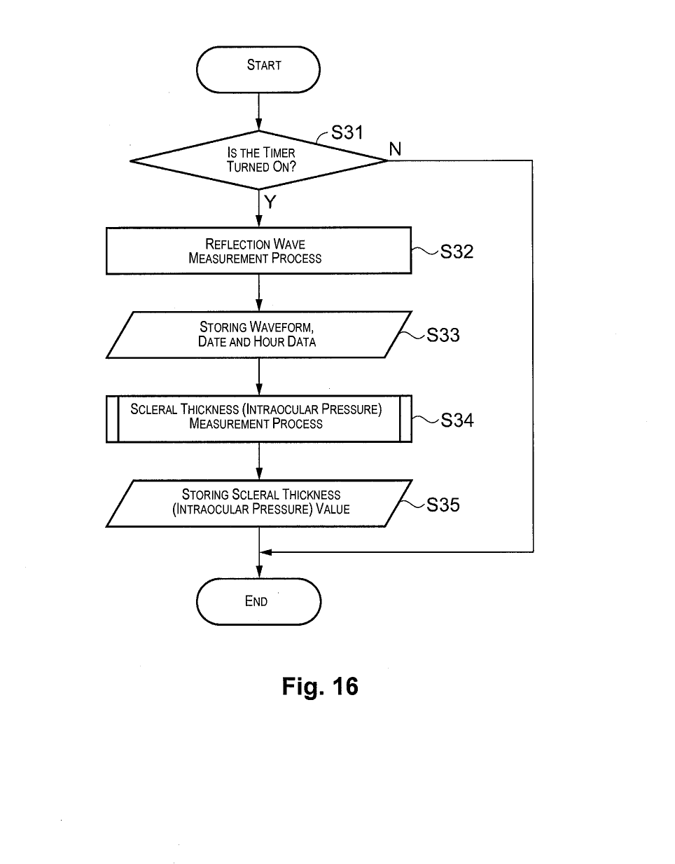

FIG. 16 is a flowchart showing the measurement process in the intraocular pressure measurement of the present embodiment. First, it judges whether or not the timer 1066 in the timer part 1065 that sent a measurement interval is on-state (Step S31). If the timer 1066 is on-state, the ultrasonic wave is transmitted from the ultrasonic sensor part 1010 in response to the measurement timing, and a reflection wave measurement process (Step S32) that measures the reflection wave from the scleral of the eyeball is performed. And, the waveform of the obtained reflection wave is stored in the primary memory 1018 with the date and hour data (Step S33). Also, in Step S31, if the timer 1066 is not on-state, the measurement process is end. By the way, the date and hour data includes the elapsed time that elapses from the wearing. Next, the measurement process of the scleral thickness or the intraocular pressure is performed in the data computing part 1040 (Step S34). And, the scleral thickness and the intraocular pressure are stored in the measurement value memory 1053, and the intraocular pressure measurement process is end. Because of this, the reflection wave from the scleral thickness of the eyeball is measured in every setting time (measurement interval) of the timer 1066.

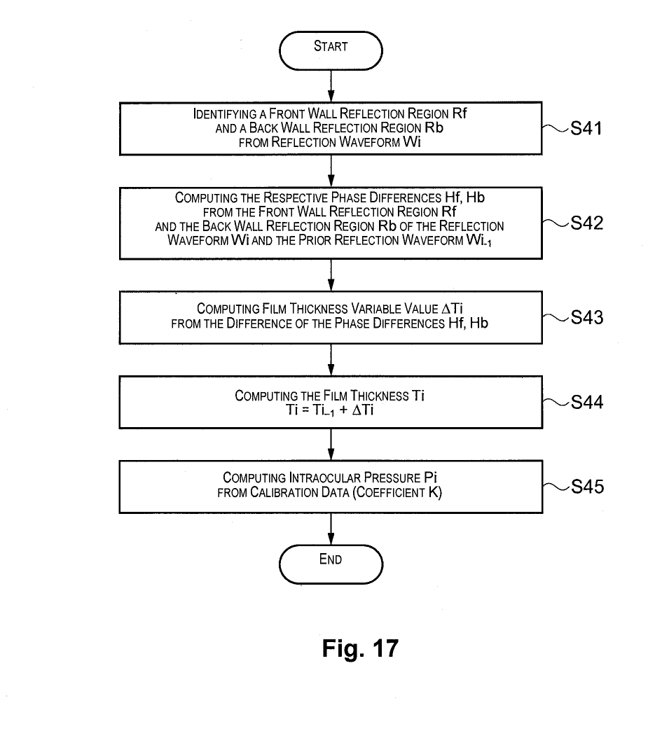

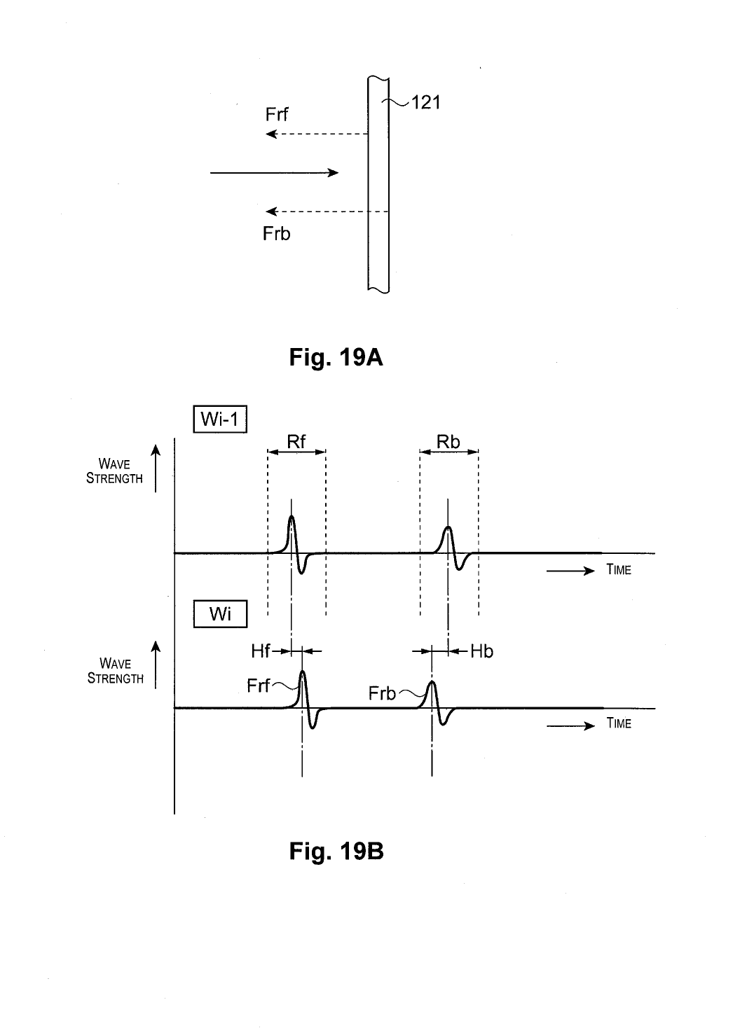

FIG. 17 is a flowchart showing a calculation process of the scleral thickness and the intraocular pressure in the intraocular pressure measurement of the present embodiment. FIGS. 19A and 19B are explanatory diagrams when a calculation process for a scleral thickness is performed. FIG. 19A is a schematic diagram showing the reflection wave reflected at the scleral, and FIG. 19B is an explanatory diagram to explain a phase difference of the reflection region and reflection wave. As shown in FIG. 17, first, the ultrasonic wave is reflected at the scleral of the eyeball. A front wall reflection region Rf of the reflection wave reflected from the front wall of the scleral, and a back wall region Rb of the reflection wave reflected from the back wall of the scleral are identified from the reflection wave Wi received in the receiving element 1011b (Step S41).

Here, as shown in FIG. 19A, a part of the ultrasonic wave transmitted to the scleral 1121 is reflected, and the reflection wave Frf reflected at the front wall of the scleral and the reflection wave Frb reflected at the back wall are generated. At this point of the reflection waveform Wi₋₁, Wi, the reflection wave Frb reflected at the back wall of the scleral 1121 is received later than the reflection wave Frf reflected at the front wall in the receiving element 1011b as shown in FIG. 19B. Also, the front wall reflection region Rf of the reflection wave reflected from the front wall of the scleral 1121 and the back wall reflection region Rb of the reflection wave reflected from the back wall of the scleral 1121 are identified from the reflection waveform Wi₋₁, Wi. By the way, Wi₋₁ is a waveform that was measured one time before the reflection waveform Wi was measured.

Next, as shown in FIG. 17, the respective phase differences Hf, Hb are computed from the front wall reflection region Rf and the back wall reflection region Rb of the reflection waveform Wi and the prior reflection waveform Wi₋₁ (Step S42). By the way, the above described processes Step S41 and Step S42 are performed in the relative variable value computing part 1041 of the data computing part 1040.

Next, the film thickness variable value ΔTi of the scleral is computed from the difference of the phase differences Hf, Hb calculated in the above processes (Step S43). The film thickness Ti of the scleral is calculated (Step S44). When Ti₋₁ indicates the thickness of the scleral in the prior calculation, the following equation will be satisfied.

Ti=Ti₋₁+ΔTi (2)

By using the equation (2), the film thickness Ti of the scleral can be calculated. By the way, the processes Step S43, Step S44 are performed in the scleral thickness variable value computing part 1043 of the data computing part 1040.

Next, the intraocular pressure Pi is calculated from the coefficient K stored in the calibration value memory 1052 (Step S45). The process of Step S45 is performed in the intraocular computing part 1044 of the data computing part 1040. The computation in the data computing part 1040 in the above process is processed by the well-known phase difference tracking method. By the way, the thickness of the scleral is calculated in Step S44, but this process can be omitted and the intraocular pressure Pi can be calculated from the film thickness variable value ΔTi of the scleral in Step S43 and the coefficient K.

In view of the discussion mentioned above, in the intraocular pressure measurement device 1001 of the present embodiment, the ultrasonic elements 1011 that tightly wear on the bottom lids 1111 covering the eyeballs are provided, and the ultrasonic wave is transmitted from the ultrasonic elements 1011 to the eyeballs. The reflection wave of the ultrasonic wave is received in the ultrasonic elements 1011 so that the intraocular pressure can be measured. In this measurement, the intraocular pressure is calculated based on the detection data stored in the data memory 1050 and the detection data detected in the ultrasonic sensor part 1010. Also, the measurement of the intraocular pressure is performed at the measurement timing set in the timer part 1065 and in the time interval. Because of this, the ultrasonic elements 1011 that are contacted to the bottom lids 1111 are provided, and the intraocular pressure can be measured in a certain measurement timing set by the timer part 1065 and in the measurement interval so that it is possible to capture the variation of the intraocular pressure easily. Also, in a certain period of time, the ultrasonic wave is transmitted and the intraocular pressure is measured intermittently so that the heat generation of the ultrasonic elements 1011 is suppressed compare to a case of the continuous measurement, and in addition, it is minimally invasive to the eyeballs. Therefore, for example, in the treatment/diagnosis of glaucoma, it is possible to perform a sensitive medication so that this can be expected to improve the effect of therapy.

Also, in the calibration value memory 1052 of the data memory 1050, the change rate data of the intraocular pressure relative to the variation of the scleral thickness of the eyeball in at least two different body postures as a calibration value is stored. It is well known that the intraocular pressure changes depending on the positions, and also, it has a correlation such that when the intraocular pressure is high, the thickness of the scleral becomes thinner, and when the intraocular pressure is low, the thickness of the scleral becomes thicker. By using the change rate data of the intraocular pressure relative to the variation of the scleral thickness of the eyeballs in the two different body postures, an absolute value of the intraocular pressure can be calculated.

### Eighth Embodiment

Next, an intraocular pressure measurement device that measures another intraocular pressure will be explained as the eighth embodiment. In the present embodiment, it configures that the ultrasonic sensor parts are contacted to the top lids, and it is the intraocular pressure measurement device that measures the intraocular pressure from the variation of the film thickness of the cornea. In the seventh embodiment, the reflection wave reflected at the scleral of the eyeball was detected, but in the eighth embodiment, the reflection wave reflected at the cornea of the eyeball is detected so that this is the different point. Therefore, the points different from the seventh embodiment will be explained.

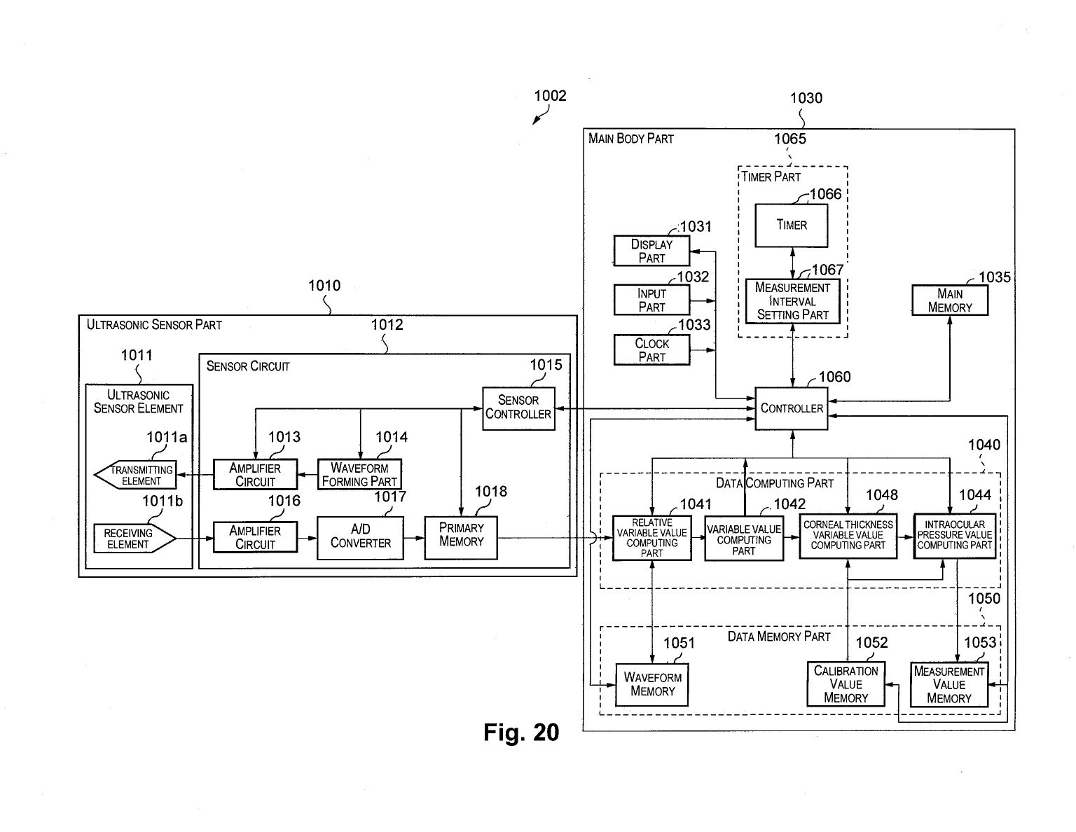

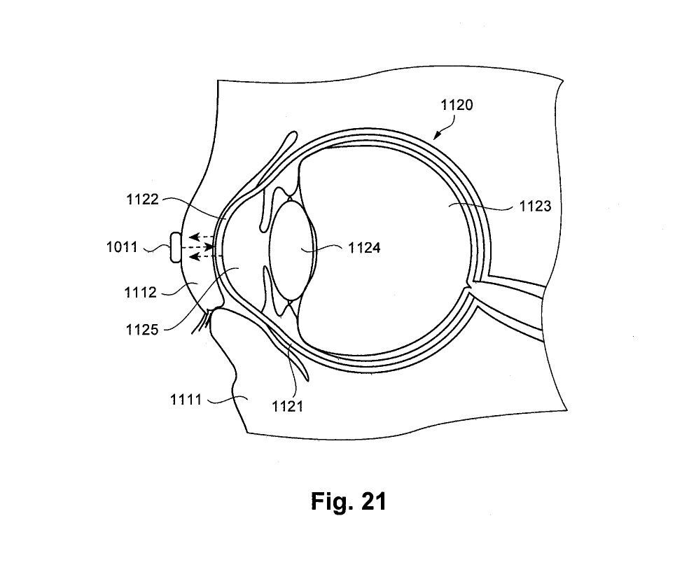

FIG. 20 is a block diagram showing a functional constitution of the intraocular pressure measurement device. FIG. 21 is a schematic cross-sectional view to explain positions of an ultrasonic sensor part, eyelid, and eyeball. As shown in FIG. 20, the waveform memory 1051, the calibration value memory 1052, and the measurement value memory 1053 are provided in the data memory 1050. In the waveform memory 1051, the waveform data of the previously received reflection waves from the front wall and the back wall of the cornea of the eyeball is stored. In the calibration value memory 1052, the respective intraocular pressures preliminary measured in at least two different postural conditions and the waveform data of the refection waves from the cornea measured in the intraocular pressure measurement device 1002 at the time of the postural condition, and the change ratio of the intraocular pressures relative to the thickness changes of the cornea are stored, and the data measured by using those data is used as a calibration value. In the measurement value memory 1053, the computed intraocular pressure value is stored.

A relative variability value computing part 1041, a variable value judgment part 1042, a corneal thickness variable value computing part 1048, and an intraocular pressure value computing part 1044 are provided in the data computing part 1040. In the relative variability value computing part 1041, the variable value of the waveform data of the reflection wave is computed from the waveform data of the reflection waves, which were received last time, from the front wall and the back wall of the cornea of the eyeball stored in the waveform memory 1051 and the waveform data of the reflection waves, which were received in this time, from the front wall and the back wall of the cornea of the eyeball stored in the primary memory 1018. In the variable value judgment part 1042, it judges whether the variable values computed in the relative variability value computing part 1041 are in a range of the defined value or out of the range of the defined value. In the corneal thickness variable value computing part 1048, the corneal thickness or the variable value of the corneal thickness is computed from the waveform data of the reflection waves stored in the calibration value memory 1052 and the variable values of the waveform data of the reflection waves computed in the relative variability value computing part 1041. In the intraocular pressure computing part 1044, an intraocular pressure value of the eyeball measured in this time is computed from the corneal thickness computed in the corneal thickness variable value computing part 1048 or the variable values of the waveform data of the reflection waves and the intraocular pressure value stored in the calibration value memory 1052. And, the computed intraocular pressure value is stored in the measurement value memory 1053.

Also, in the present embodiment as shown in FIG. 21, the ultrasonic element 1011 is tightly placed on the top eyelid 1112. The ultrasonic wave is generated from the ultrasonic element 1011, and when it reaches to the cornea 1122, reflection waves are reflected at the front wall and the back wall of the cornea. By detecting the time lag of receiving time of the reflection waves, the thickness of the cornea 1122 can be computed.

In view of the discussion mentioned above, the measurement of the intraocular pressure is performed by using the reflection waves from the cornea in the present embodiment. The point that the reflection wave is reflected from the cornea is different from the point that the reflection wave is reflected from the scleral in the seventh embodiment. This point is only the difference, and it can obtain the same effect as the seventh embodiment. By the way, in the seventh embodiment and the eighth embodiment, the intraocular pressure measurement devices 1001, 1002 that measure an intraocular pressure were discussed, but it is possible to measure axial length, depth of the anterior chamber, lens thickness, and the like as an eyeball biological information collection device.

The invention is not limited to the embodiments described above, and the specific configurations and procedures in the practice of the invention can be appropriately modified to other configurations in the scope that achieves the advantage of the invention. And, many modifications are possible by a person of ordinary skill in the art in the technical idea of the invention. The modification examples are discussed below.

### Modification Example 1

In the first embodiment, the ultrasonic transmitter 17 and the ultrasonic receiver 18 and the sensor circuit 19 are provided on the same surface in the circuit board 16 of the ultrasonic sensor part 8. The ultrasonic transmitter 17 and the ultrasonic receiver 18 and the sensor circuit 19 can be provided on a different surface. Also, a through electrode can be formed on the circuit board 16, and the ultrasonic transmitter 17 and the ultrasonic receiver 18 and the sensor circuit 19 can be connected electrically. The area of the circuit board 16 can be minimized. Or, the area of the ultrasonic transmitter 17 and the ultrasonic receiver 18 can be widened so that the receiving sensitivity can be improved.

### Modification Example 2

In the first embodiment, the ultrasonic sensor part 8 has a configuration that the element substrate 23 is superimposed on the circuit board 16, and the vibrating membrane 24 is provided in the opening 16a on the element substrate 23. And, the vibrating membrane 24 has a beam structure in both ends. However, this is not only the structure. A concave portion can be formed on the circuit board 16 as an opening 16a, and a vibrating membrane 24 can be provided on the concave portion. Even in this structure, the vibrating membrane 24 has a beam structure in both ends. In these two structures, one that has a structure to be easily manufactured can be selected.

### Modification Example 3

In the first embodiment, the sensor supporting part 3f is a metal having elasticity, but it can be a resin including filler. A desired shape can be formed. Also, the sensor supporting part 3f can be a hollow tubular shape. And, a wire can be set in the tubular. In addition, the wire can be provided inside part of the sensor supporting part 3. The degrees of the freedom for the exterior design can be increased.

### Modification Example 4

In the first embodiment, the intraocular pressure computing part 52 computed the intraocular pressure. Also, the intraocular pressure that is accumulated relative to time can be calculated. An extent of damage to the eyeball 12 can be calculated by the intraocular pressure.

### Modification Example 5

In the first embodiment, the base part 8a of the ultrasonic sensor part 8 is fixed on the sensor supporting part 3f. The sensor supporting part 3f and the base part 8a can be rotatably connected. The ultrasonic sensor part 8 is oriented toward the bottom lid 7 of the subject so that the ultrasonic sensor part 8 can be tightly contacted to the bottom lid 7 of the subject easily. Also, in the fourth embodiment, the sensor supporting part 77f and the base part 8a can be rotatably connected.

### Modification Example 6

In the fourth embodiment, the intraocular pressure was calculated by measuring the thickness of the cornea 12b, but also, it can be calculated by measuring the thickness of the lens 12d or measuring a dimension of the eyeball 12. It can be utilized in the treatment of various eye diseases.

1. An eyeball biological information collection device that is arranged to be worn by a subject, comprising:
an ultrasonic sensor part being configured to transmit an ultrasonic wave to an eyeball of the subject and receive a reflection wave of the ultrasonic wave reflected within the eyeball at a time of use of the eyeball biological information collection device; and
a pressing part being configured to press the ultrasonic sensor part to eyelid of the subject at the time of use.

2. The eyeball biological information collection device according to claim 1, wherein
the ultrasonic sensor part includes
a substrate in which first and second openings are arranged in an array pattern,
first and second ultrasonic elements being formed at the first and second openings respectively,
wherein
the first ultrasonic element includes a first vibrating membrane being configured to cover the first opening and a first piezoelectric element part being configured in the first vibrating membrane, and
the first piezoelectric element part includes a first lower electrode being configured on the first vibrating membrane, a first piezoelectric body film being configured to cover at least a part of the first lower electrode, and a first upper electrode being configured to cover at least a part of the first piezoelectric body film, and
the second ultrasonic element includes a second vibrating membrane being configured to cover the second opening and a second piezoelectric element part being configured in the second vibrating membrane, and
the second piezoelectric element part includes a second lower electrode being configured on the second vibrating membrane, a second piezoelectric body film being configured to cover at least a part of the second lower electrode, and a second upper electrode being configured to cover at least a part of the first piezoelectric body film.

3. The eyeball biological information collection device according to claim 2, wherein the substrate is a semiconductor substrate.

4. The eyeball biological information collection device according to claim 3, further comprising
an amplifier circuit being configured to amplify a received signal, wherein
the ultrasonic sensor part is provided integrally with the first and second ultrasonic elements and the amplifier circuit.

5. The eyeball biological information collection device according to claim 4, wherein
the ultrasonic sensor part includes an ultrasonic receiver to which the first piezoelectric element part and the second piezoelectric element part are connected in series.

6. The eyeball biological information collection device according to claim 5, wherein
the ultrasonic sensor part includes an ultrasonic transmitter which includes third and fourth ultrasonic elements being formed at third and fourth openings of the substrate respectively,
wherein
the third ultrasonic element includes a third vibrating membrane being configured to cover the third opening and a third piezoelectric element part being configured in the third vibrating membrane, and
the third piezoelectric element part includes a third lower electrode being configured on the third vibrating membrane, a third piezoelectric body film being configured to cover at least a part of the third lower electrode, and a third upper electrode being configured to cover at least a part of the third piezoelectric body film, and
the fourth ultrasonic element includes a fourth vibrating membrane being configured to cover the fourth opening and a fourth piezoelectric element part being configured in the fourth vibrating membrane, and
the fourth piezoelectric element part includes a fourth lower electrode being configured on the fourth vibrating membrane, a fourth piezoelectric body film being configured to cover at least a part of the fourth lower electrode, and a fourth upper electrode being configured to cover at least a part of the first piezoelectric body film, and
the third piezoelectric element part and the fourth piezoelectric element part are connected in parallel.

7. The eyeball biological information collection device according to claim 1, further comprising
a gelatinous ultrasonic conductor being configured on a side of the ultrasonic sensor part facing toward the eyelid.

8. The eyeball biological information collection device according to claim 4, further comprising
an A/D converter being configured to convert a signal, which is outputted from the amplifier circuit, to a digital signal, and
a memory part being configured to store the digital signal.

9. The eyeball biological information collection device according to claim 1, wherein
the pressing part includes an elastic member made of an elastic material, and
a part of the elastic member is arranged to be in contact with a head region of the subject.

10. An eyeball biological information collection device that is used by wearing to a subject, comprising:
an ultrasonic sensor part being configured to transmit an ultrasonic wave to an eyeball of the subject and receive a reflection wave of the ultrasonic wave reflected at the eyeball at a time of use of the eyeball biological information collection device; and
an elastic member being configured on a side, which is an opposite side facing toward an eyelid of the subject, at the time of use of the ultrasonic sensor part.

11. An eyeball biological information collection device that is arranged to be worn by a subject, comprising:
an ultrasonic sensor part being configured to transmit an ultrasonic wave to an eyeball of the subject and receive a reflection wave reflected at the eyeball at a time of use of the eyeball biological information collection device; and
an elastic supporting member being configured to support the ultrasonic sensor part and extending in a direction toward an eyelid of the subject at the time of use.

12. An eyeball biological information collection device that is arranged to be worn by a subject, comprising:
an ultrasonic sensor part being configured to transmit an ultrasonic wave to an eyeball of the subject and receive a reflection wave of the ultrasonic wave reflected at the eyeball at a time of use of the eyeball biological information collection device;
a frame being arranged to be worn onto an ear and nose of the subject at the time of use; and
a supporting part being made of an elastic material that is attached to the frame, and configured to support the ultrasonic sensor part in a direction toward an eyelid of the subject at the time of use.

13. An eyeball biological information collection device that is arranged to be worn by a subject, comprising:
an ultrasonic sensor part being configured to transmit an ultrasonic wave to an eyeball of the subject and receive a reflection wave reflected at the eyeball at a time of use of the eyeball biological information collection device;
a winding part being wound on a head region of the subject at the time of use; and
a pressing part being made of an elastic material that is located between the winding part and the ultrasonic sensor part, and configured to press the ultrasonic sensor part to the eyelid of the subject.

14. An eyeball biological information collection device that is arranged to be worn by a subject, comprising:
an ultrasonic sensor part being configured to transmit an ultrasonic wave to an eyeball of the subject and receive a reflection wave of the ultrasonic wave reflected at the eyeball at a time of use;
an contacting part contacting tightly the ultrasonic sensor part to the eyelid of the subject at the time of use;
a data computing part being configured to compute eyeball biological information based on detection data detected in the ultrasonic sensor part;
a data memory part being configured to store the detection data detected in the ultrasonic sensor part and computation data computed in the data computing part;
a timer part being configured to set a measurement timing and a measurement interval based on time information; and
a controller being configured to control the ultrasonic sensor part, the data computing part, the data memory part, and the timer part;
the data computing part being configure to compute the biological information of the eyeball based on the detection data detected at the measurement timing and the measurement interval.

15. The eyeball biological information collection device according to claim 14, wherein
the ultrasonic sensor part includes
a substrate in which first and second openings are arranged in an array pattern,
first and second ultrasonic elements being formed at the first and second openings respectively,
wherein
the first ultrasonic element includes a first vibrating membrane being configured to cover the first opening and a first piezoelectric element part being configured in the first vibrating membrane, and
the first piezoelectric element part includes a first lower electrode being configured on the first vibrating membrane, a first piezoelectric body film being configured to cover at least a part of the first lower electrode, and a first upper electrode being configured to cover at least a part of the first piezoelectric body film, and
the second ultrasonic element includes a second vibrating membrane being configured to cover the second opening and a second piezoelectric element part being configured in the second vibrating membrane, and
the second piezoelectric element part includes a second lower electrode being configured on the second vibrating membrane, a second piezoelectric body film being configured to cover at least a part of the second lower electrode, and a second upper electrode being configured to cover at least a part of the first piezoelectric body film.

16. The eyeball biological information collection device according to claim 15, further comprising
an amplifier circuit being configured to amplify a received signal, wherein
the ultrasonic sensor part is provided integrally with the first and second ultrasonic elements and the amplifier circuit.

17. The eyeball biological information collection device according to claim 14, wherein
the data computing part includes a relative variable value computing part being configured to compute a variable value based on last detection data detected in the ultrasonic sensor part, and a variable value judgment part being configured to judge computation data of variable value computed in the relative variable value computing part.

18. The eyeball biological information collection device according to claim 14, wherein
the data memory part has a calibration value memory that stores a calibration value, and
the calibration value memory has eyeball biological information measured in at least two different body postures.

19. The eyeball biological information collection device according to claim 18, wherein
the calibration value memory of the data memory part has change rate data of an intraocular pressure relative to a thickness variation of a scleral measured in the two different body postures as the calibration value, and
the data computing part has a film thickness variable value computing part being configured to compute thickness variation of the scleral of the eyeball based on detection data detected in the ultrasonic sensor part, and an intraocular pressure computing part being configured to compute the intraocular pressure from the thickness variation of the scleral of the eyeball computed in the film thickness variable value computing part.

20. The eyeball biological information collection device according to claim 18, wherein the calibration value memory of the data memory part has change rate data of an intraocular pressure relative to a corneal thickness variation of eyeball measured in the two different body postures as a calibration value, and wherein the data computing part has a corneal thickness variable value computing part that computes corneal thickness variation of the eyeball based on detection data detected in the ultrasonic sensor part, and an intraocular pressure computing part that computes the intraocular pressure from the corneal thickness variation of the eyeball computed in the corneal thickness variable value computing part.

21. An eyeball biological information collection method for obtaining eyeball biological information in a state in which an ultrasonic sensor part is worn on a head region of a subject, the method comprising:
transmitting and receiving an ultrasonic wave for an eyeball in a predetermined measurement timing and a predetermined measurement interval from the ultrasonic sensor part that is contacted on an eyelid of the subject; and
computing the eyeball biological information based on a detection data detected in the ultrasonic element.

22. The eyeball biological information collection method according to claim 21, wherein
the computing the eyeball biological information includes computing the eyeball biological information based on the detection data and the preliminary obtained eyeball biological information measured in at least two different body postures.

23. The eyeball biological information collection method according to claim 21, further comprising:
computing thickness variation of a scleral of the eyeball based on the detection data; and
computing an intraocular pressure from the thickness variation of the scleral of the eyeball based on a preliminary obtained reflection wave data from the scleral of the eyeball in at least two different body postures and the intraocular pressure value.

24. The eyeball biological information collection method according to claim 21, further comprising:
computing corneal thickness variation of the eyeball based on the detection data; and
computing the intraocular pressure from the corneal thickness variation of the eyeball based on a preliminary obtained reflection wave data from the cornea of the eyeball in at least two different body postures and the intraocular pressure value.
